SEQUENCE LISTING (2 sequences)

SEQ ID NO: 1 - DNA, 20 bp, Artificial sequence
  Synthetic
agcgagcatcccccaaagtt

SEQ ID NO: 2 - DNA, 20 bp, Artificial sequence
  Synthetic
gggcacgaaggctcatcatt

(19) United States
(12) Patent Application Publication
(10) Pub. No.: US 2023/0405145 A1
(43) Pub. Date: Dec. 21, 2023

(54) IRON OXIDE NANOPARTICLE FOR SUPPRESSING DRUG-RESISTANT GENE FOR THE TREATMENT OF GLIOBLASTOMA

(71) Applicant: University of Washington, Seattle, WA (US)

(72) Inventors: Miqin Zhang, Seattle, WA (US); Richard G. Ellenbogen, Seattle, WA (US); Kui Wang, Seattle, WA (US); Forrest M. Kievit, Seattle, WA (US); John R. Silber, Seattle, WA (US); Zachary Stephen, Seattle, WA (US); Peter A. Chiarelli, Seattle, WA (US)

(73) Assignee: University of Washington, Seattle, WA (US)

(21) Appl. No.: 18/248,139

(22) PCT Filed: Oct. 6, 2021
(86) PCT No.: PCT/US2021/053753

§ 371 Date: Apr. 6, 2023

Related U.S. Application Data
(60) Provisional application No. 63/089,403, filed on Oct. 8, 2020.

(51) Int. Cl. A61K 47/69; A61K 47/62; A61K 47/34; C12N 15/113; A61P 35/00

(52) U.S. Cl. CPC A61K 47/6923; A61K 47/62; A61K 47/6925; A61K 47/34; C12N 15/1137; A61P 35/00

(57) ABSTRACT

A nanoparticle for targeted siRNA delivery comprising an iron oxide core and a chitosan-polyethylene glycol-polyethylenimine copolymer coating surrounding the core, chlorotoxin covalently coupled to the coating, and an siRNA reversibly associated to the coating by non-covalent interaction. Methods for making the nanoparticle and methods for using the nanoparticle to suppress the expression of O⁶-methylguanine-DNA methyltransferase (MGMT), treating brain cancer, and killing cancer stem cells.

### CROSS-REFERENCE TO RELATED APPLICATION

This application claims the benefit of U.S. Application No. 63/089,403, filed Oct. 8, 2020, expressly incorporated herein by reference in its entirety.

### STATEMENT OF GOVERNMENT LICENSE RIGHTS

This invention was made with Government support under R01 CA161953 and R01 EB026890 awarded by National Institutes of Health. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 3915-P1224WOUW_Seq_List_FINAL_20211006_ST25.txt. The text file is 1 KB; was created on Oct. 6, 2021; and is being submitted via EFS-Web with the filing of the specification.

### BACKGROUND OF THE INVENTION

Glioblastoma multiforme (World Health Organization grade IV; GBM) is the most common and lethal primary brain tumors in adults and has the lowest 5-year survival rate (<5%) among all cancers. Despite the treatment advances made over the last decades, the prognosis for GBM remains dismal, with most patients succumbing within 9 to 15 months_ENREF_2. The inclusion of the alkylating agent temozolomide (TMZ) with radiotherapy extends median survival by 2.5 months, the first substantial increase in survival observed for GBM patients in 40 years; currently TMZ is the contemporary standard of care for GBM_ENREF_3. Nevertheless, this improved clinical outcome has been shadowed by the limited responsiveness of the majority of GBMs to TMZ, largely attributed to a DNA repair protein, O⁶-methylguanine-DNA methyltransferase (MGMT), that removes the TMZ-induced DNA lesions. Attempts to suppress MGMT activity have been made by systemic administration of a chemical MGMT inhibitor, O⁶-benzylguanine (BG), to interrupt DNA repair, but have achieved limited success primarily because of the low serum solubility, poor BBB penetration, and short serum half-life of BG. More importantly, BG produces severe myelotoxicity in combination with TMZ. Nanoparticle (NP) formulations have been developed for BG delivery to circumvent these limitations, but they have been limited to in vitro studies and the intra-tumoral infusion by convection-enhanced delivery. Systemic injection of nanocarrier-transported inhibitors, which is a preferred administration method in clinical practice, has gained minimal success because of the inability to deliver a sufficient amount of the inhibitor. The difficulty in delivery of an efficacious amount of a drug while maintaining acceptable systemic toxicity is common to all NP formulations designed to systemic drug delivery and has been the major impediment in NP-based chemotherapy for treating solid tumors.

Systemic administration of small interfering RNA (siRNA) based MGMT (siMGMT) inhibitors may hold better therapeutic potential than the delivery of chemical MGMT inhibitors such as BG because siRNA can selectively silence disease-causing genes and only a small number of siRNA molecules are needed to attain the efficacious dose._ENREF_15 However, a major challenge in clinical application of siRNAs for cancer therapy is the protection of siRNAs from degradation by serum during trafficking in the bloodstream. For treating GBM, the blood-brain barrier (BBB) imposes an additional challenge, as the BBB impedes the access of foreign substances including therapeutics to the brain.

Despite the advances in the development of therapeutic agents for the treatment of GBM, a need exists for improved therapeutic agents and delivery systems that improve standard-of-care therapy. The present invention seeks to fulfill this need and provides further related advantages.

### SUMMARY OF THE INVENTION

In one aspect, the invention provides a nanoparticle for targeted siRNA delivery.

In certain embodiments, the nanoparticle comprises:
(a) an iron oxide core and a chitosan-polyethylene glycol-polyethylenimine copolymer coating surrounding the core;
(b) chlorotoxin covalently coupled to the coating; and
(c) an siRNA reversibly associated to the coating by non-covalent interaction.

In other embodiments, the nanoparticle comprises:
(a) an iron oxide core and a chitosan-polyethylene glycol-polyethylenimine copolymer coating surrounding the core;
(b) chlorotoxin covalently coupled to the coating; and
(c) an siRNA reversibly associated to the coating by non-covalent interaction.

In further aspects, the invention provides methods for using the nanoparticle as described herein.

In certain embodiments, the invention provides a method for suppressing the expression of O⁶-methylguanine-DNA methyltransferase (MGMT) in a subject, comprising administering an effective amount of a nanoparticle as described herein to a subject in need thereof.

In other embodiments, the invention provides a method for treating brain cancer in a subject, comprising administering a therapeutically effective amount of a nanoparticle as described herein to a subject in need thereof.

In further embodiments, the invention provides a method for killing cancer stem cells in a subject, comprising administering a therapeutically effective amount of a nanoparticle as described herein to a subject in need thereof.

The nanoparticle of the invention can be used diagnostically. In certain embodiments, the invention provides a method for magnetic resonance imaging a tumor, for example, to detect tumor growth rate and monitor the response of the tumor to treatment, in a subject. In these embodiments, an effective amount of a nanoparticle as described herein is administered to a subject to be imaged.

In another aspect of the invention, methods for making siRNA-containing nanoparticles are provided. The method for making the siRNA-containing nanoparticles is versatile and facilitates the production of nanoparticles having selected targeting agents and their complementary siRNAs.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIGS. 1A-1G illustrate the design and characterization of representative nanoparticles of the invention: NP-siRNA-CTX. FIG. 1A is a schematic illustration of NP-siRNA-CTX synthesis process. FIG. 1B is the proton NMR analysis of CP and CP-PEI showing the successful incorporation of PEI onto CP. Both characteristic peaks of the —O—CH2-CH2- group of PEG (peak I) and —NH2-CH2-CH2- group of PEI (highlighted peak II) are present in the CP-PEI spectrum. All samples were analyzed in D₂O. FIG. 1C verifies the presence of CTX on NP by SDS gel electrophoresis (arrow: CTX bands). Release of CTX by thioether linkage cleavage of purified (Lanes 1 & 2) and unpurified (Lanes 3 & 4) NP-siRNA-CTX. NP-siRNA-CTX in Lane 1 & 3 were denatured before gel electrophoresis. FIGS. 1D and 1E are TEM images showing the size and morphology of (d) iron oxide core (1D) and polymer-coated iron oxide core (1E). Scale bar=50 nm. FIG. 1F compares hydrodynamic size (left panel) and zeta potential (right panel) distributions of NP, NP-siRNA, and NP-siRNA-CTX measured by dynamic light scattering. FIG. 1G presents the gel retardation assay of NP:siRNA at various weight ratios of NP to siRNA from 0.5 to 20, showing NP protects siRNA from degradation by serum nucleases. Top panel: siRNA products of NP-siRNA-CTX incubated with heparin in the absence of serum showing the release of siRNA, confirming NP binds siRNA by electrostatic interaction; middle panel: siRNA products of NP-siRNA-CTX incubated in the absence of both heparin and serum showing no detectable siRNA (i.e., not released); bottom panel: siRNA products of NP-siRNA-CTX incubated with in serum containing heparin showing the release of siRNA.

FIGS. 2A-2J illustrate the internalization of NP-siRNA and biological activity of NP-siMGMT in human GBM cell lines. FIG. 2A is a schematic representation illustrating the mechanism of NP-mediated knockdown of MGMT expression. FIG. 2B are fluorescence images of SF763 cells acquired at various time points after treatment with NP-siRNA, showing localizations of endosome, NP, and siRNA. Nuclei were stained with Hoechst 33342. Scale bar=20 m. FIGS. 2C and 2D compare SF763 MGMT mRNA content (FIG. 2C) and MGMT activity (FIG. 2D) of cleared cell homogenates 72 hr after treatment with NP-siGFP or NP-siMGMT (untreated cells were used as a control; *P<0.01). FIG. 2E shows the viability of MGMT-proficient SF763 cells, assayed by clonogenic assay 48 hr after treatment with NP-siMGMT as compared to untreated cells. FIG. 2F are fluorescence images acquired by confocal laser scanning microscopy of DAPI stained nuclei and MGMT in untreated (left), NP-siGFP-treated (middle), and NP-siMGMT-treated (right) SF763 cells 72 hr after the treatment. White arrows indicate MGMT expression in nuclei. Scale bar=20 m. FIGS. 2G-2I are survival curves (survival rate vs. drug dose) of SF763 cells treated with NP-siGFP, NP-siMGMT, or untreated, and then treated with either TMZ (FIG. 2G), Dox (FIG. 2H), or Oxaliplatin (FIG. 2I). FIG. 2J shows the survival of MGMT-deficient A1235 GBM cells when they were treated with NP-siMGMT and then with TMZ. All survival curve results are the mean±SD of 9 determinations from three independent experiments.

FIGS. 3A-3N illustrate biodistribution, BBB penetration, and toxicity of NP-siRNA-CTX (siScr-Dy677-CTX) assessed in C57BL/6J WT mice. FIG. 3A compares fluorescence images of the organs of mice treated by intravenous injection of NP-siRNA-CTX showing the distributions of in liver, kidneys, and spleen, assessed 2 hr (second row) and 48 hr (third row) post injection using an IVIS Lumina II system. Images of untreated mice (first row) are also shown for reference. FIG. 3B compares fluorescence intensities in liver, kidneys, and spleen, quantified from the images shown in FIG. 3A; data are mean±SD of signals from 4 mice. FIG. 3C are fluorescence images showing the accumulation and retention of NP-siRNA-CTX in the brains of mice at 2 hr and 48 hr post injection. FIG. 3D are fluorescence images of mouse brain sections, acquired by confocal laser scanning microscopy at 10 min and 6 hr post injection, showing distribution of NP-siRNA-CTX, as indicated by arrows and dotted circles, in brain sections. Here, nuclei were stained with DAPI and endothelial cells with anti-CD31 antibody labeled with Alexa 488. Scale bar=20 μm. FIGS. 3E-3N summarizes the results of in vivo toxicity studies of a NP-siRNA-CTX. Twenty-four hours after injection, NP-siMGMT-CTX had negligible effect on blood white cell count (WBC) (FIG. 3E), hemoglobin (HGB) (FIG. 3F), hematocrit (HCT) (FIG. 3G), platelet count (PLT) (FIG. 3H), albumin (FIG. 3I), alanine transaminase (ALT) (FIG. 3J), aspartate transaminase (AST) (FIG. 3K), alkaline phosphatase (ALP) (FIG. 3L), bilirubin (FIG. 3M), and blood urea nitrogen (BUN) (FIG. 3N). All values are mean±SD of determinations made in 5 mice.

FIGS. 4A-4E illustrate that NP-siMGMT-CTX sensitizes GBM6 xenograft cells expressing GSC markers to TMZ. FIG. 4A are fluorescent images of tumor sections harvested from mice bearing GBM6 xenografts. Tumor tissue sections were immunostained to show nuclei (left column); and various GSC markers (middle column). The third column is the overlay of first and second columns. As shown, tumor tissues showed the expressions of GSC markers including CD44 (first row), Nestin (second row), and Sox-2 (third row). The white dash line outlines the tumor boundary. Scale bar=20 μm. FIGS. 4B and 4C are Xenogen IVIS fluorescence image of mouse brains and confocal fluorescence images of brain tumor sections, respectively, from mice bearing GBM6 tumors and administered with NP-siRNA-CTX via tail vail injection (n=3 per condition). The image was acquired 3 hr post NP-siRNA-CTX injection. Mice bearing tumors and receiving no injection served as control (untreated). NP-siRNA-CTX was seen to be in brain (FIG. 4B) and accumulate in tumor cells with nuclei stained blue (FIG. 4C) indicating NP-siRNA-CTX specifically target tumor. Scale bar=100 μm. FIG. 4D compares MGMT and 3-actin protein expression of tumor sections of GBM6-bearing mice 24 hr after treatment with either NP-siGFP-CTX or NP-siMGMT-CTX or left untreated (top: Western blot bands for 3-actin and MGMT proteins; bottom: MGMT protein expression evaluated from the Western blot band intensities and the data were normalized to 3-actin protein. As shown, the NP-siMGMT-CTX treatment significantly reduced MGMT protein expression as compared to the treatment with NP-siGFP-CTX and untreated condition. FIG. 4E illustrates MGMT activity of GBM6 intracranial tumors of the mice shown in FIG. 4D, showing that the NP-siMGMT-CTX treatment reduced MGMT activity as compared to the treatment with NP-siGFP and untreated condition.

FIGS. 5A-5E illustrate that NP-siMGMT-CTX increases TMZ sensitivity and prolongs survival. FIG. 5A is a schedule of tumor inoculation and treatment. Mice were injected orthotopically with GBM6 cells at day 0, and four weeks later (day 28), xenograft-bearing mice received four daily intravenous injections of NP-siMGMT-CTX and four daily administration of TMZ by oral gavage starting one day (day 29) after the first injection of NP-siMGMT-CTX. FIG. 5B compares T2-weighted MRI images of representative brains of mice (n=4) showing tumor progression from week 2 after tumor inoculation to week 7 for untreated mice (rows 1 and 2), mice treated with NP-siGFP-CTX+TMZ (rows 3 and 4), and mice treated with NP-siMGMT-CTX+TMZ (rows 5 and 6) mice. The tumor volume, estimated from the 3 largest orthogonal dimensions, is represented by the side bar in the bottom left of the brain image for weeks 5, 6, and 7. Arrows mark the first appearance of tumor. The last column on the right shows optical images of H&E stained whole brain sections obtained at sacrifice. FIG. 5C compares tumor volumes (mean±SD) in mice treated under three different conditions (n=4 per condition) as a function of time, evaluated from MR images shown in FIG. 5B. The black arrow indicates the time point of the onset of the treatment. FIG. 5D is a flow cytometry analysis of TMZ-induced tumor cell death assessed by double immunostaining for the apoptosis marker Annexin V and the GSC marker CD44. The NP-siMGMT-CTX+TMZ treatment caused GCS apoptosis 5.2- and 2.9-fold higher than the untreated condition and the treatment with NP-siGFP-CTX+TMZ, respectively. FIG. 5E illustrates Kaplan-Meier survival curves of untreated mice, and mice treated with either NP-siGFP-CTX, NP-siMGMT-CTX, or free TMZ (n=8 for each treatment condition), showing significantly prolonged survival for mice treated with siMGMT+TMZ. P values were calculated by the log-rank test. Results are from 3 independent experiments using 3 mice for each treatment.

FIG. 6 compares survival curves (viability vs. drug dose) of GBM6 cells treated with NP-siGFP in which siGFP is complexed on NP-PEI, NP-siMGMT in which siMGMT is complexed onto NP-PEI, or left untreated. All results are presented as the mean±SD from three independent experiments using the Alamar blue viability assay.

FIG. 7 compares survival curves (viability vs. drug dose) of GBM6 cells treated with NP-PEI-siGFP covalent, NP-siMGMT covalent, or left untreated. siGFP and siMGMT are covalently bound to the nanoparticles with a PEI coating following the method reported in our paper (Biomaterials, 32 (24), 5717-5725 (2011). All results are presented as the mean±SD from three independent experiments using the Alamar blue viability assay.

FIG. 8 compares viability of GBM6 cells treated with various nanoparticle formulations including (as shown on horizonal axis) NP-PEI-siMGMT complex, NP-PEI-siMGMT covalent, NP-pArg-siMGMT covalent, NP-pLys-siMGMT covalent and control cells (non-treatment: cells receiving no NP treatment, scramble siRNAs: cells treated with scramble siRNA). In NP-PEI-siMGMT complex, siMGMT was complexed onto NP-PEI. Scramble siRNAs refers to the siRNAs with the molecular weight similar to siMGMT but with different gene sequence and are used as a negative control. In NP-PEI-siMGMT covalent, NP-pArg-siMGMT covalent, and NP-pLys-siMGMT covalent, siMGMTs are covalently bonded on NP coated with polyethyleneimine (PEI), polyarginine (pArg), polylysine (pLys) coatings, respectively.

### DETAILED DESCRIPTION OF THE INVENTION

The present invention provides a nanoparticle for targeted siRNA delivery. In certain embodiments, the nanoparticle comprises an iron oxide core and a chitosan-polyethylene glycol-polyethylenimine copolymer coating surrounding the core, chlorotoxin covalently coupled to the coating, and an siRNA reversibly associated to the coating by non-covalent interaction. Methods for making the nanoparticle and methods for using the nanoparticle to suppress the expression of O⁶-methylguanine-DNA methyltransferase (MGMT), treating brain cancer, and killing cancer stem cells are provided.

In one aspect, the invention provides a nanoparticle for targeted siRNA delivery.

In certain embodiments, the nanoparticle comprises:
(a) an iron oxide core and a chitosan-polyethylene glycol-polyethylenimine copolymer coating surrounding the core;
(b) chlorotoxin covalently coupled to the coating; and
(c) an siRNA reversibly associated to the coating by non-covalent interaction.

In other embodiments, the nanoparticle comprises:
(a) an iron oxide core and a chitosan-polyethylene glycol-polyethylenimine copolymer coating surrounding the core;
(b) chlorotoxin covalently coupled to the coating; and
(c) an siRNA reversibly associated to the coating by non-covalent interaction.

In certain embodiments, the nanoparticles of the invention include chlorotoxin as a targeting agent useful for targeting brain tumors with the appropriate siRNA. It will be appreciated that in other embodiments, the nanoparticles of the invention do not include chlorotoxin but do include a targeting agent to direct the nanoparticle to the cell of interest. In these embodiments, the siRNA is selected to reduce the expression of an oncogene or expression of a treatment-resistance gene in the cell of interest target by the nanoparticle (treatment resistance can be resistance from radiation, chemotherapy, and immune therapy treatments).

In certain embodiments, the nanoparticles of the invention that include chlorotoxin target brain cancer tumor cells. As described and demonstrated herein, these nanoparticles are effective passing the blood brain barrier (BBB) and are effective in treating brain cancers.

The nanoparticle of the invention includes an siRNA reversibly associated with the nanoparticle's coating. As used herein, the term “reversibly associated to the coating by non-covalent interaction” means that the siRNA is not covalently coupled to the nanoparticle (e.g., not covalently coupled to the coating). The term “non-covalent interaction” refers to an associative interaction between the siRNA and the nanoparticle (i.e., the nanoparticle's coating) by ionic interactions (e.g., between cationic sites of the coating and anionic sites of the siRNA), and not by covalent bonding (i.e., the formation of a covalent bond between the coating and the siRNA by, for example, a direct bond or through a linker). In the nanoparticle, the siRNA is associated to the nanoparticle's coating through a non-covalent interaction with the polyethylenimine (PEI) of the coating. The siRNA is stably associated with the nanoparticle as the nanoparticle trafficks the circulatory system and is advantageously released from the nanoparticle once the nanoparticle reaches its cellular target (endosome) where the pH of the cell's endosome is effective to facilitate release of the siRNA from the nanoparticle. The nanoparticle of the invention differs from other similarly constituted known nanoparticles in which an siRNA is covalently coupled to the nanoparticle and results in ineffective release of the siRNA from the nanoparticle in environment of the endosome. The advantages of the nanoparticle of the invention having the siRNA reversibly associated with the coating (nanoparticle) by non-covalent interaction is described in detail below.

The nanoparticles of the invention advantageously have a high positive charge to facilitate release of the siRNA from the nanoparticle in the endosomes of the cells targeted by the nanoparticle. In certain embodiments, the nanoparticle has a zeta potential from about 10 my to about 30 mv. Typically, nanoparticles having high charge are often very toxic to cells and tissues. However, the nanoparticles of the invention are not due to the chitosan-PEG of the coating, which reduces nanoparticle toxicity to cells and tissues.

To make the claimed NPs, IOSPM (iron oxide nanoparticles with a siloxane poly(ethylene glycol) (PEG) monolayer) are conjugated with CP-PEI to provide the nanoparticle's coating, which is then treated with the siRNA.

The iron oxide (IO) particle that forms the core of the nanoparticle of the invention has a size (diameter) that ranges from about 4 to about 12 nm. The size of IOSPM ranges from about 6 to about 15 nm.

The chitosan-PEG (CP) useful in the nanoparticle of the invention is prepared from chitosan having a molecular weight from about 500 to about 20,000 dalton and from polyethylene glycol having a molecular weight from about 500 to about 5000 dalton.

The polyethylenimine (PEI) useful in the nanoparticle of the invention has a molecular weight from about 2000 to about 40,000

The chitosan-PEG-PEI (CP-PEI) useful in the nanoparticle of the invention includes from about 10 to about 50 percent by weight chitosan-PEG.

In certain embodiments, the nanoparticle's iron oxide core has a size from about 4 to about 12 nm. In certain embodiments, the nanoparticle has a size from about 35 to about 80 nm.

In certain embodiments, the chlorotoxin is present in an amount from 1 to about 200 chlorotoxins per nanoparticle.

In certain embodiments, the siRNA is present in an amount from about 100 to about 400 siRNAs per nanoparticle. In certain embodiments, the siRNA reduces the expression of O⁶-methylguanine-DNA methyltransferase (MGMT) in a subject. In certain of the embodiments, the siRNA is siMGMT. As used herein “siMGMT” defines a family of siRNAs that reduce the expression of MGMT. It will be appreciated that various types of siMGMT are commercially or otherwise available and differ by their specific nucleic acid sequence.

In another aspect of the invention, a pharmaceutical composition is provided. The pharmaceutical composition comprises a nanoparticle as described herein and a pharmaceutically acceptable carrier. In certain embodiments, the pharmaceutical composition is formulated for intravenous injection. Representative pharmaceutically acceptable carriers suitable for intravenous injections include saline and dextrose solutions.

In further aspects, the invention provides methods for using the nanoparticle as described herein.

In certain embodiments, the invention provides a method for suppressing the expression of O⁶-methylguanine-DNA methyltransferase (MGMT) in a subject, comprising administering an effective amount of a nanoparticle as described herein to a subject in need thereof.

In other embodiments, the invention provides a method for treating brain cancer in a subject, comprising administering a therapeutically effective amount of a nanoparticle as described herein to a subject in need thereof. In certain of these embodiments, the brain cancer is a glioblastoma. In certain of these embodiments, the brain cancer is glioblastoma multiforme (GBM).

In further embodiments, the invention provides a method for killing cancer stem cells in a subject, comprising administering a therapeutically effective amount of a nanoparticle as described herein to a subject in need thereof.

In certain embodiments, the nanoparticle of the invention can be used diagnostically. In certain embodiments, the invention provides a method for magnetic resonance imaging a tumor, for example, to monitor tumor growth rate and to monitor the response of the tumor to treatment, in a subject. In these embodiments, an effective amount of a nanoparticle as described herein is administered to a subject to be imaged.

In certain embodiments of the methods described above, the nanoparticle is administered intravenously. In certain embodiments of the methods, the subject is a human.

In another aspect of the invention, methods for making siRNA-containing nanoparticles are provided. The method for making the siRNA-containing nanoparticles is versatile and facilitates the production of nanoparticles having selected targeting agents and their complementary siRNAs. The method relies on the use of a base iron oxide nanoparticle comprising an iron oxide core and a chitosan-polyethylene glycol-polyethylenimine copolymer coating surrounding the core. Once the targeting agent and complementary siRNA are selected for a particular, the appropriate targeting agent can be covalently coupled to the base nanoparticle and the siRNA can be reversibly associated to the nanoparticle by non-covalent interaction.

In certain embodiments, the method for making a nanoparticle for targeted siRNA delivery comprises contacting an siRNA with an iron oxide nanoparticle, wherein the iron oxide nanoparticle comprises an iron oxide core and a chitosan-polyethylene glycol-polyethylenimine copolymer coating surrounding the core, and a targeting agent covalently coupled to the coating. As described below, in certain embodiments the targeting agent is chlorotoxin (targeting brain cancers) and the siRNA is an siRNA that reduces the expression of O⁶-methylguanine-DNA methyltransferase (MGMT).

The following is a description of representative embodiments of the nanoparticle of the invention, and representative methods for making and using the nanoparticle.

Design and Physicochemical Properties of NP-siMGMT-CTX

A representative nanoparticle of the invention (“NP-siRNA-CTX”) consists of an iron oxide (Fe₃O₄) core coated with a chitosan-PEG-PEI co-polymer that is further conjugated with siRNA and a GBM targeting ligand chlorotoxin (CTX). With the intrinsic superparamagnetism of the iron oxide nanocore, NP-siRNA-CTX can also serve as an MRI contrast agent for non-invasive tumor imaging and treatment response monitoring. Iron oxide nanoparticles are biodegradable and are used clinically for MRI contrast enhancement. _ENREF_41 Chitosan is a biocompatible and biodegradable polycationic polysaccharide with ample functional groups for attachment of subsequent biomolecules. PEG is grafted to chitosan to serve as a stabilizer that prevents nanoparticle agglomeration. PEI binds and protects siRNA and facilitates endosome escape after cellular internalization. _ENREF_18CTX, a 36-amino acid peptide, can target the majority of primary brain tumors. CTX also facilities BBB penetration by receptor-mediated transcytosis. The clinical safety and applicability of CTX have been demonstrated in early phase clinical trials in which ¹³¹I-conjugated CTX was used to treat recurrent GBMs.

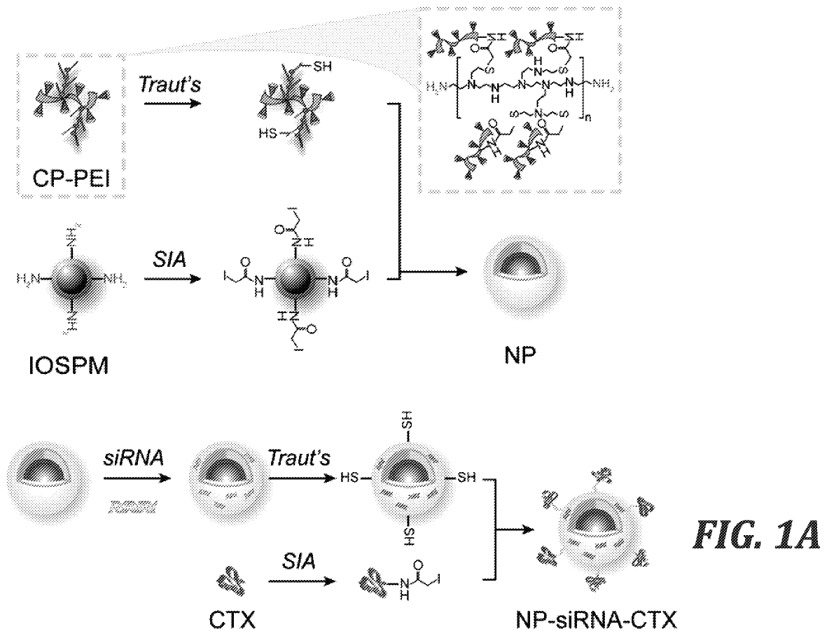

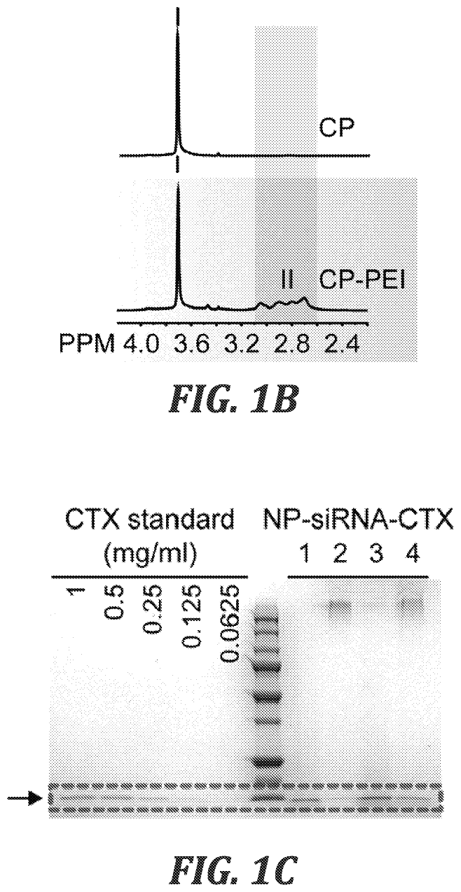

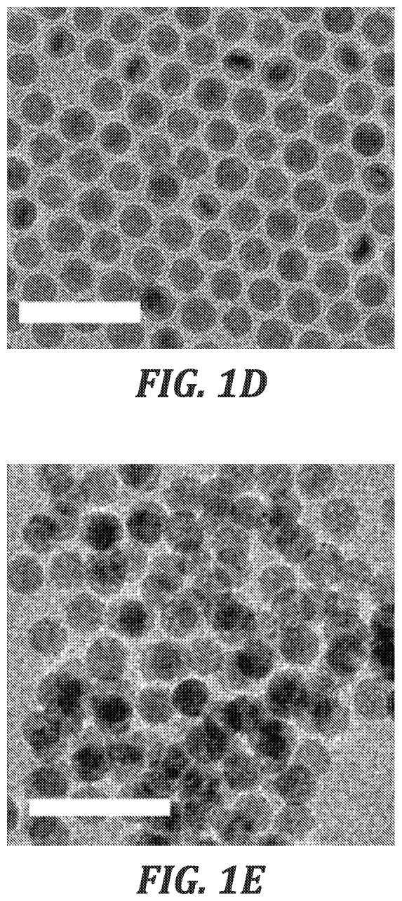

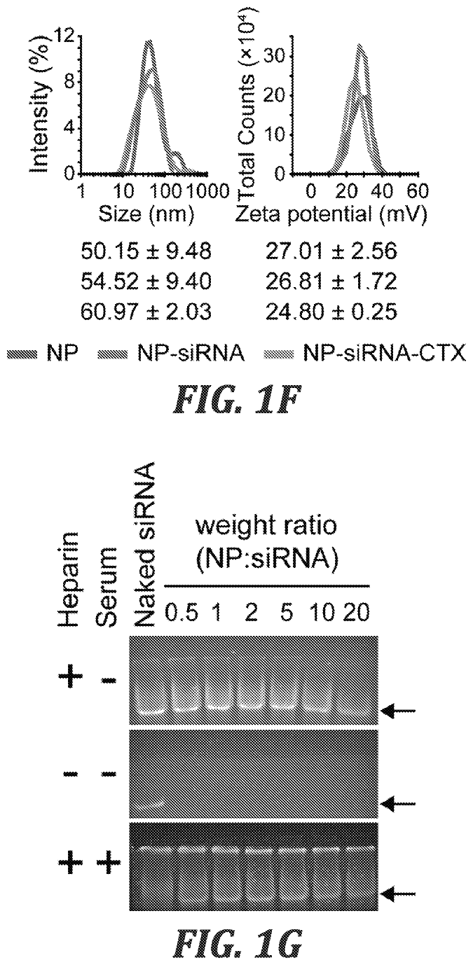

NP-siRNA-CTX was synthesized by covalent attachment of chitosan-PEG-PEI to an iron oxide core bearing NH₂ groups (IOSPM) through a two-step process (see FIG. 1A). First, PEG is grafted to chitosan by a method described previously (N. Bhattarai, H. R. Ramay, J. Gunn, F. A. Matsen, M. Zhang, Journal of Controlled Release 2005, 103, 609) to form chitosan-PEG (CP). The reactive amine groups of PEI are modified with Traut's agent to render a free thiol group; the IOSPM is concurrently modified with N-succinimidyl-iodoacetate (SIA). The thiolated PEI is then covalently attached to SIA-modified IOSPM to form the base nanoparticle (“NP”). Second, NP is complexed with siRNAs at various weight ratios (Fe equivalent of NP to siRNA) to form NP:siRNA (here, siRNA is used as a general term and represents either siGFP or siMGMT). CTX is conjugated to NP:siRNA using the heterobifunctional linker (SIA) and Traut's agent to form NP:siRNA-CTX. Covalent bonding of CP-PEI to NP was examined by proton NMR (1H-NMR), which showed a characteristic peak of the ethylene group of PEG at 3.7 ppm and a characteristic peak of the ethylenimine (—NH—CH₂CH₂—) repeat unit of PEI at 2.6-3.1 ppm (see FIG. 1B). The binding of CTX with CP was confirmed by SDS-PAGE (see FIG. 1C). The hydrodynamic size and zeta potential are key determinants of the pharmacological efficacy of the nanomaterials. TEM imaging revealed that the spherical morphology and small size (10-12 nm) of the iron oxide core (see FIG. 1D) was retained after conjugation with CP-PEI copolymer (see FIG. 1E). The dynamic light scattering analysis revealed that the hydrodynamic diameter of siRNA-bound NP (NP-siRNA, blue curve) at a 1:2 ratio of iron to siRNA (corresponding to about 300 siRNA molecules per NP) was 54.52±9.40 nm (see FIG. 1F, left panel), similar to that of NP without siRNA, indicating that siRNA binding did not alter the NP size which is well within the range that helps evade the rapid clearance by the reticuloendothelial system. Little difference in surface charge was observed between NPs with or without bound siRNA (27.01±2.56 mV vs. 26.81±1.72 mV), which were both in the positive region (see FIG. 1F, right panel). The value of the surface charge is seen similar to those observed for other NP formulations. FIG. 1F also shows that the binding of CTX had little effect on the size and zeta potential of the NP.

To evaluate the ability of the NP to protect siRNAs from degradation by serum nucleases and investigate the siRNA binding mechanism, the release of siRNAs from the NP-siRNAs was assessed by the gel retardation assay. NP-siRNA samples of various weight ratios of NP to siRNA were prepared and the samples of each ratio was divided into three groups. One group of samples were treated with heparin (electrostatic disrupting agent) in the absence of serum; a second group of samples (control) were incubated in buffer without heparin and serum; and a third group of samples were treated with both serum and heparin. The reaction products were then loaded in agarose gel wells without purification. siRNA that remained bound to the NP would stay in the top portion of the well while unbound siRNA would migrate towards the bottom of the well (see FIG. 1G). Incubation of NP-siRNA in heparin released siRNA over a wide range of amounts of siRNA loaded on NP (i.e., NP:siRNA=0.5-20) (see FIG. 1G, top row). No siRNA bands were detected in the absence of heparin and serum (see FIG. 1G, middle row), indicating that siRNA was bound to NP by electrostatic interaction, which suggests protection of the siRNA from external molecules including the ethidium bromide used to stain the gel. siRNAs bands were seen when NP-siRNA was incubated in serum before adding heparin, (see FIG. 1G, bottom row), confirming the protection of siRNAs by NP against serum nuclease. It is also noted that no changes in the physical size and morphology of the NP core and in the hydrodynamic size (54.83±2.85 nm) of NP-siRNA were observed after incubation with serum. Together, these results indicate that our NP protects siRNAs from nuclease degradation and the siRNA binding on NP was solely mediated by electronic interaction with PEI.

Biological Activities of NP-siMGMT In Vitro

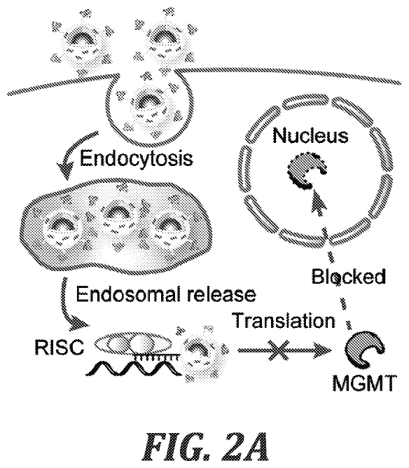

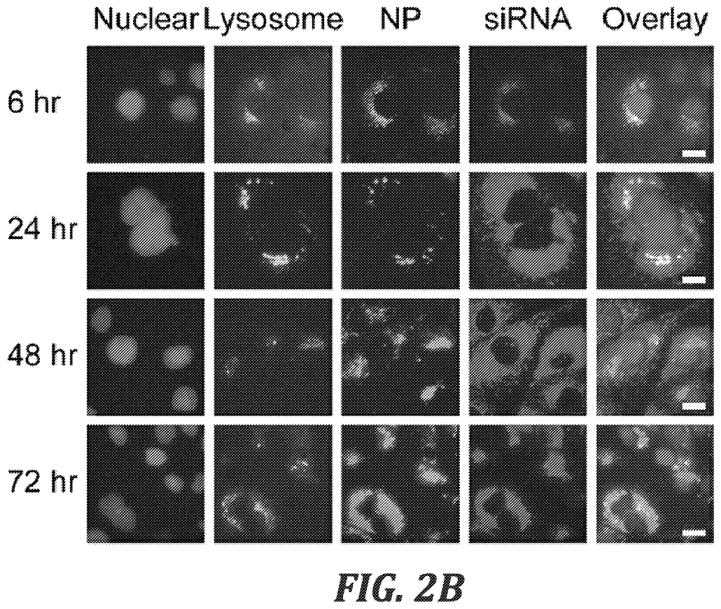

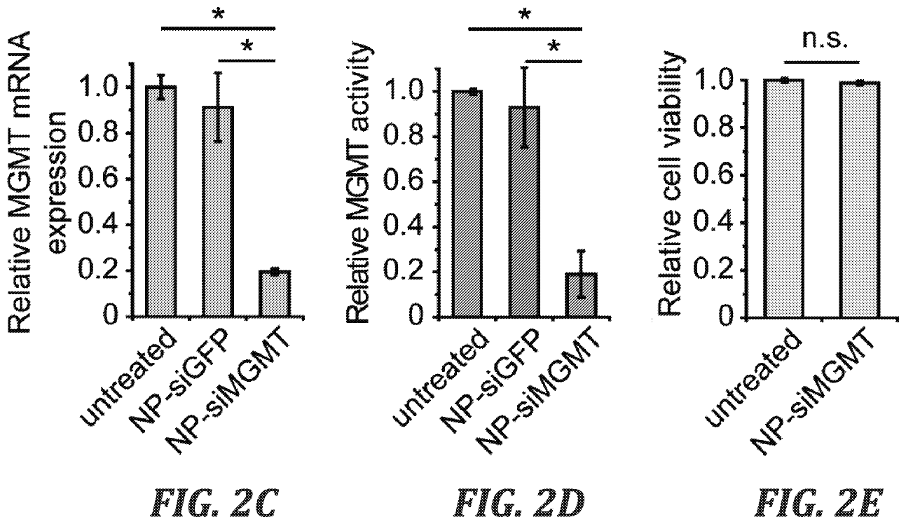

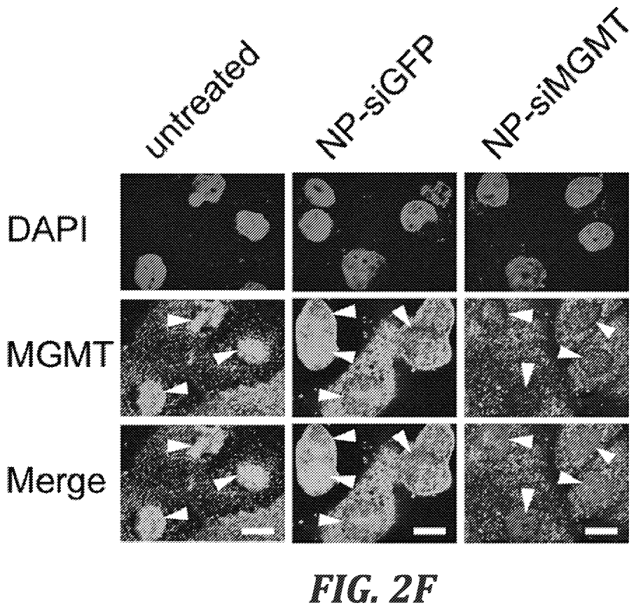

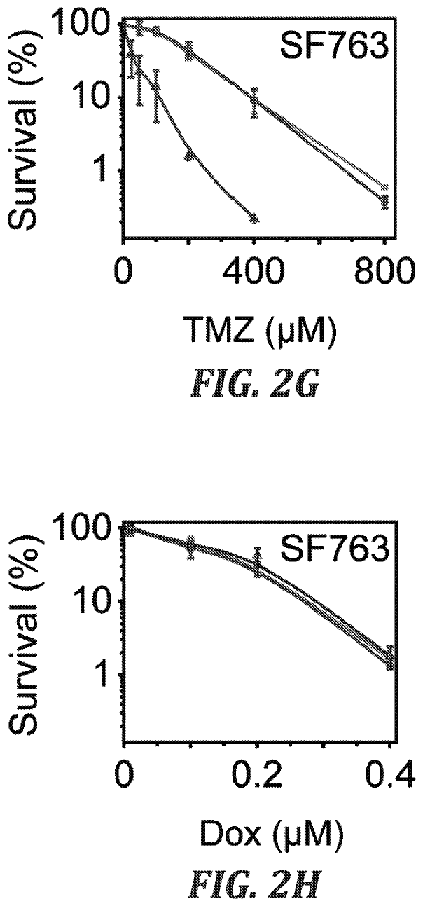

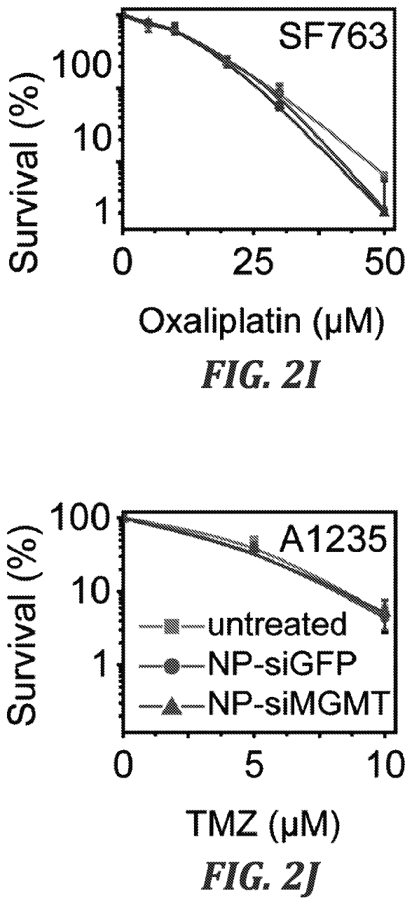

NPs loaded with siRNA against MGMT (NP-siMGMT) to suppress MGMT expression in glioma cells, which would result in the increased sensitivity of the glioma cells to TMZ, was examined. SF763 was used as the model cell line for this study. SF763 is a human GBM-derived cell line characterized by high MGMT expression and high resistance to TMZ. _ENREF_9 FIG. 2A conceptually illustrates the mechanism of NP-mediated siMGMT delivery and suppression of MGMT. NPs are taken up by cells by endocytosis, escape endosomes by the proton-sponge effect from the tertiary amines of PEI on the surface coating, and release siRNAs in cytoplasm. The siRNAs in the cytoplasm react with the RNA-induced silencing complex (RISC), which binds to and cleaves messenger RNA (mRNA) to prevent its translation into MGMT protein.

The in vitro study was performed in two steps: first, the cellular internalization and intracellular trafficking of NPs was examined with fluorescence imaging to reveal the underlying mechanism, and then the efficacy of NP-siMGMT in sensitizing GBM cells to TMZ by the clonogenic assay was evaluated. These NPs without containing CTX tumor targeting ligand do not provide a significant increase in NP internalization during normal in vitro cell culture conditions.

To study the cellular internalization and intracellular trafficking, SF763 cells were treated with NPs labeled with cyanine 3 and loaded with a scrambled siRNA (NP-siRNA) that was labeled with Dy677 siRNA. The cells treated with NP-siRNA were incubated with LysoTracker Green probe to label the endosomes of the cells. Cell nuclei were labeled with Hoechst 33342. Fluorescence images of the cells were acquired at various time points up to 72 h after NP-siRNA treatment (see FIG. 2B). As shown, the co-localization of the endosome, NP, and siRNA at 6 hrs after the treatment indicated that NP-siRNA entered the endosomes, and NP and siRNA remained complexed. After 24 hrs, the fluorescent signal of siRNA shifted away from the signals of the NP and endosome and spread out, indicating the siRNA had been released from the NPs and escaped from the endosomes. The NPs were also seen to exit from the endosomes and accumulated in the cytoplasm 48 hrs after the treatment. Additionally, the fluorescence signal of siRNA faded out at 72 h, indicating the degradation or elimination of the released siRNA. These results demonstrated the ability of our NP to circumvent intracellular barriers, deliver and release siRNA into GBM cells. To assess if NP-siRNA can effectively knockdown MGMT in vitro, SF763 cells were treated with either NP-siMGMT or NP-siGFP (i.e., siRNA against GFP as a control) for 72 hr and the MGMT mRNA content and activity of the treated cells are shown in FIGS. 2C and 2D, respectively. Untreated cells were also included as a control in the study. Both the MGMT expression and activity of cells treated with NP-siMGMT declined approximately 5-fold compared to cells treated with control NP-siGFP. This was corroborated by immunocytochemistry analysis where the substantial reduction in MGMT fluorescence signal strength for NP-siMGMT treated cells was observed, both in the nucleus where MGMT functions to remove methylguanine groups and in the cytoplasm (see FIG. 2E). To examine if the NP caused any toxicity, SF763 cells were treated with NP-siMGMT for 48 hrs and cell viability was studied by clonogenic assay. Notably, incubation of SF763 cells with NP-siMGMT alone had little effect on cell viability, indicating minimal intrinsic cytotoxicity of NP-siMGMT (see FIG. 2E).

The efficacy of NP-siMGMT in suppressing the resistance of SF763 cells to TMZ treatment was evaluated to confirm whether the function of NP-siMGMT is specific to MGMT expressing cells or TMZ using the clonogenic assay. SF763 cells were incubated with NP-siMGMT or NP-siGFP (control) for 48 hr and then incubated for another 24 hrs with TMZ at various concentrations. Cells were cultured in fresh medium that did not contain NPs or TMZ for 10 days to allow colony formation. Cytotoxicity was quantified by linear regression analysis of plots of log surviving fraction vs TMZ dose to obtain LD₅₀ (lethal dose at 50% cell death). Untreated cells were also included in this study as a control. As shown by the survival curves in FIGS. 2G, NP-siMGMT treated SF763 cells were more sensitive to TMZ as evidenced by >13-fold reduction in LD₅₀ compared to untreated cells (green curve) (14 μM vs. 183 μM; P≤0.05) and NP-siGFP-treated cells (14 μM vs. 193 μM; P≤0.05). Similar experiments were conducted with Dox (doxorubicin) and Oxaliplatin, rather than TMZ, as therapeutic agents (see FIGS. 2H and 2I, respectively) to investigate if NP-siMGMT would increase the sensitivity of SF763 cells to these drugs. Both Dox and Oxaliplatin are DNA damaging agents, but unlike TMZ, do not produce the methylguanine adducts that MGMT repairs. Dox intercalates DNA and prevents the function of topoisomerase, and Oxaliplatin, like other platinum-based drugs, generates interstrand crosslinks and DNA-protein crosslinks that MGMT is unable to repair. As shown, NP-siMGMT had no apparent effect _ENREF_22 on alternating the sensitivity of SF763 cells to oxaliplatin or doxorubicin. To see if NP-siMGMT has any effect on a non-MGMT expressing GMB cell line, the same experiment with TMZ as therapeutic agent was conducted on A1235 cells (a non-MGMT expressing cell line). FIG. 2J shows no apparent difference in cytotoxicity for untreated, NP-siGFP, and NP-siMGMT+TMZ (LD₅₀ of 4.7 μM vs. 3.6 μM vs. 3.4 μM; P≤0.05), indicating that NP-siMGMT did not sensitize A1235 cells to TMZ. These data indicate that NP-siMGMT can introduce biologically active siRNA into GBM cells to sensitize them to TMZ and significantly reduce drug resistance.

To evaluate the efficacy of NP-siMGMT in suppressing the resistance of another cell line of GBM against TMZ, GBM stem-like cells (GBM6) were first transfected with either NP-siGFP or NP-siMGMT for 48 hr and TMZ was then applied to the cells and incubated for another 48 hr. Based on the survival rate data, the untreated GBM6 cells displayed strong TMZ resistance as the cells remained nearly 90% viable after being treated with 4 mM TMZ for 48 hr. GBM6 cells treated with NP-siMGMT remained only 44% viable after 48-hour treatment with 4 mM TMZ, which is significantly lowered than that of the untreated and the NP-siGFP-treated GBM6 cells (P<0.05). These results indicate that NP-siMGMT in combination with TMZ was able to more effectively kill GBM6 cells.

Biodistribution, BBB Penetration, and Toxicity of a NP-siRNA-CTX In Vivo

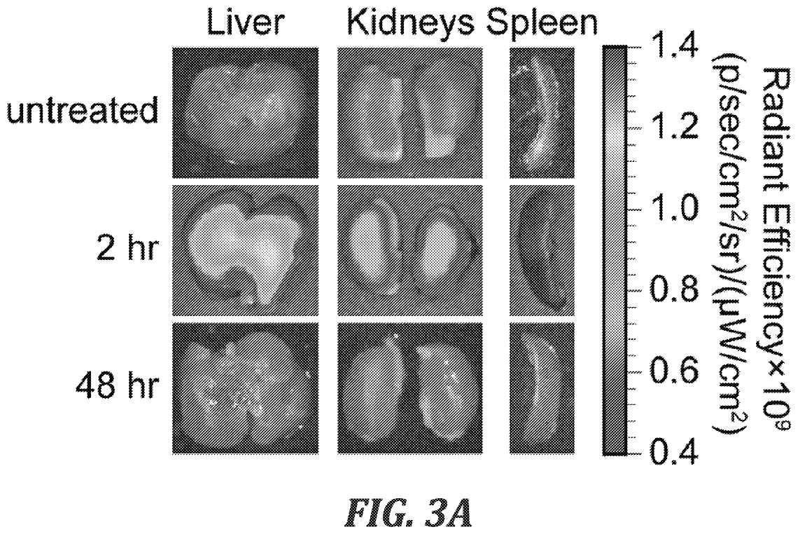

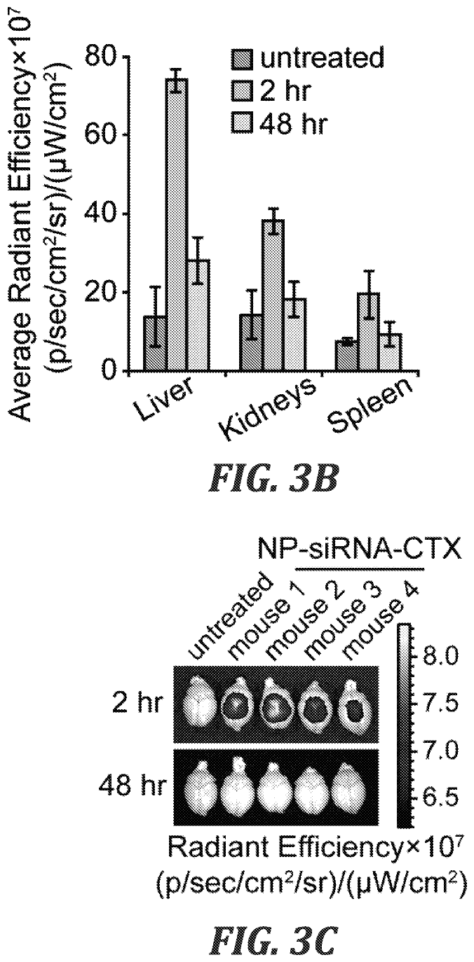

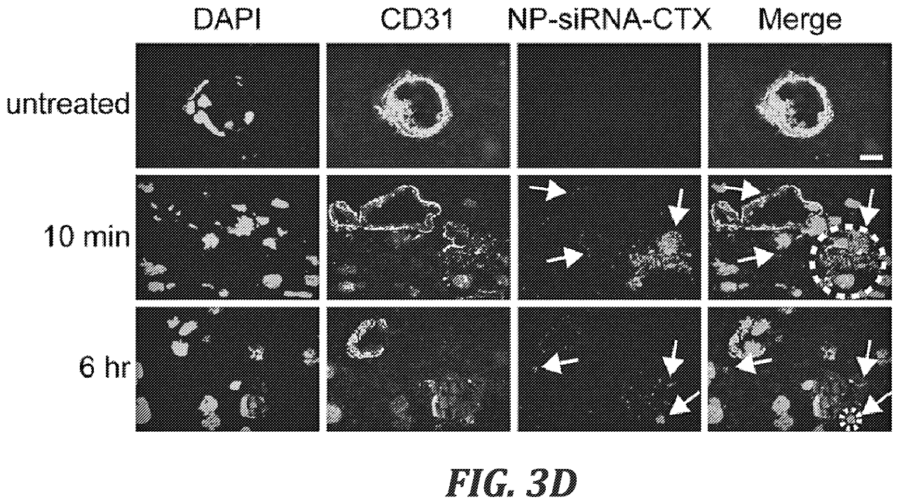

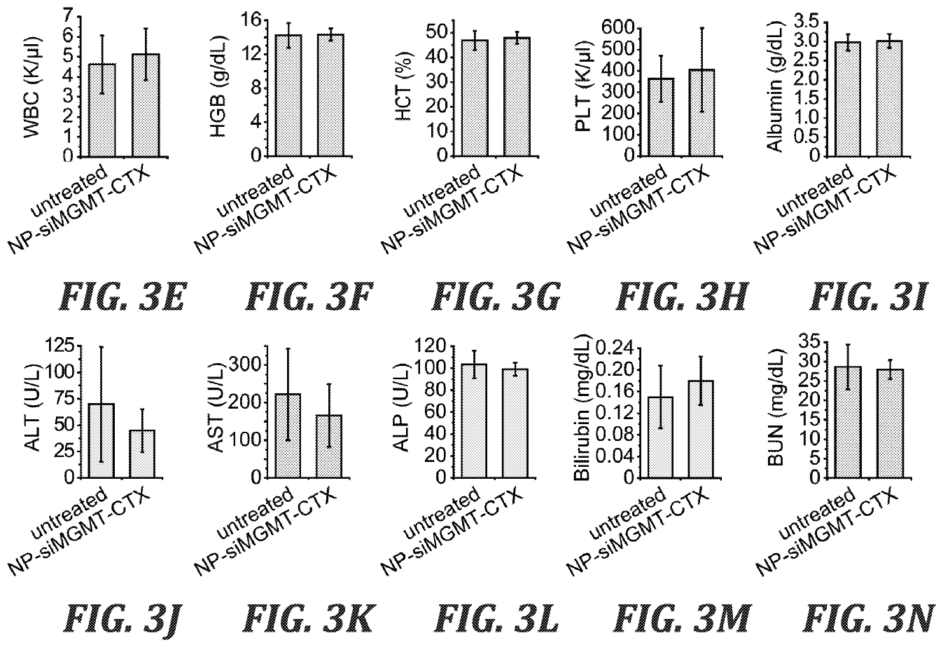

To characterize biodistribution, an NP bound with a scrambled siRNA that was conjugated with the Dy677 fluorophore (NP-siScr-Dy677-CTX) (“NP-siRNA-CTX”) was constructed. NP-siRNA-CTX in wild-type mice (C57BL/6J WT) was administered via tail vein injection. Organs were collected before, 2 hr, and 48 hr post injection, and the fluorescence was quantified to determine siRNA distribution as described previously (M. J.-E. Lee, O. Veiseh, N. Bhattarai, C. Sun, S. J. Hansen, S. Ditzler, S. Knoblaugh, D. Lee, R. Ellenbogen, M. Zhang, J. M. Olson, PLoS ONE 2010, 5, e9536). As shown in FIG. 3A, the fluorescence signal at 2 hr after the administration was localized predominantly in liver with lesser amounts in kidneys and spleen (major organs of elimination). By 48 hr, fluorescence in these organs (FIG. 3B), brain (FIG. 3C), and blood had returned to the levels of those of untreated mice. These data indicate that the majority of NP-siRNA-CTX is eliminated from the body within 48 hr and does not persist in healthy organs.

To determine whether NP-siRNA-CTX was able to penetrate the BBB, we administered NP-siRNA-CTX systemically in the C57BL/6J WT mice (which have an intact BBB). Brain tissues were collected at 10 min and 6 hr post administration and examined by confocal microscopy for the localization of the NP (Dy677) and CD31 (an endothelial cell marker). Fluorescence images of brain sections (FIG. 3D) showed that NP-siRNA-CTX (red) accumulated inside blood vessels (CD31) at 10 min after NP administration and escaped from the blood vessels and accumulated in brain tissues 6 hr post administration, indicating that NP-siRNA-CTX penetrated the BBB.

To examine the toxicity of the NP, C57BL/6J WT mice were injected daily with NP-siMGMT-CTX for 4 days. Blood was collected at the 24 hr after the last injection and tested for a panel of serum markers (10) diagnostic for myelo-, hepato-, and renal-toxicity; mice receiving PBS injections (untreated) served as control (FIGS. 3E-3N). As shown, no substantial difference in the levels of these markers were observed between mice injected with NP:siMGMT-CTX and control mice. These findings suggest that repeated treatment with NP-siMGMT-CTX did not perturb hematopoiesis or hepatic and renal function. Gross examination by H&E staining of sectioned tissues also revealed no changes to the integrity of heart, liver, lung, spleen, and kidneys from treated mice, indicating that NP-siMGMT-CTX is not grossly cytotoxic.

NP-siRNA-CTX Targets Tumors and Suppresses MGMT Activity in Orthotopic Serially-Passaged GBM Patient-Derived Xenografts Displaying GBM Stem Cell (GSC) Markers

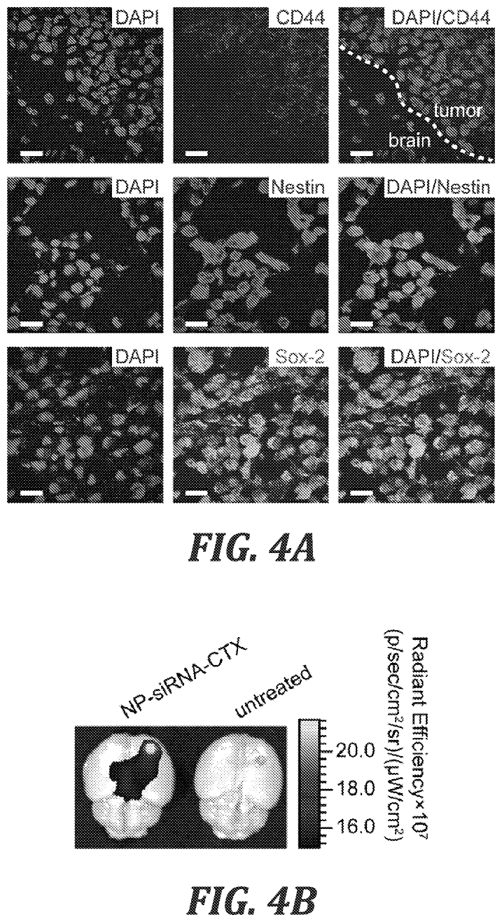

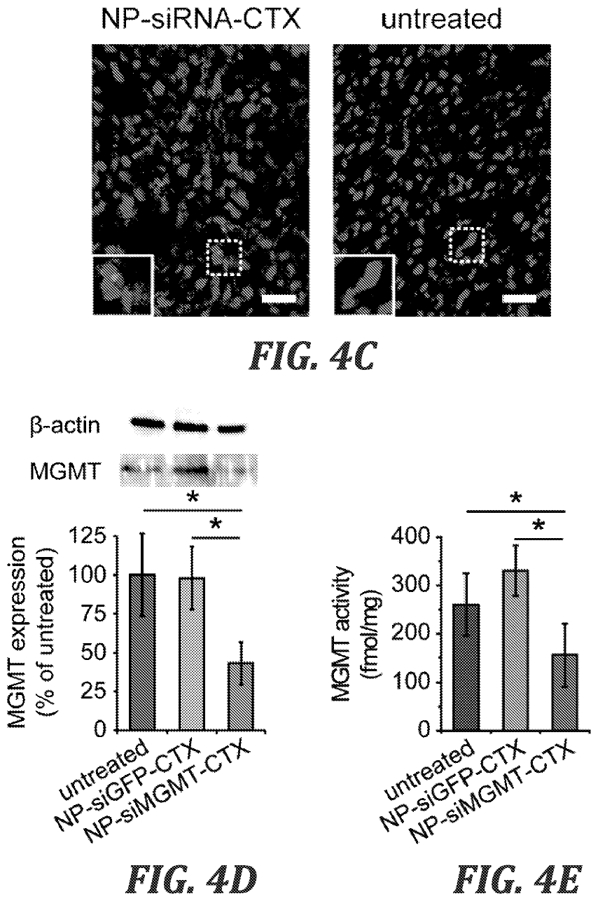

The stem-like GBM cells (GSCs) are recognized to contribute to tumor aggressiveness and recurrence and be responsible for the tumor's resistance to TMZ treatment. The activity of the NPs was examined in a widely accepted orthotopic glioblastoma serially-passaged patient-derived xenograft mouse model that displays many of the histopathologic features that characterize GBM or recapitulate biological hallmarks of GBM_ENREF_36. This extensively characterized GBM6 (GSC) cell model can form intracranial tumors in nude mice. GBM6 expresses a high level of MGMT and contains a high fraction of GSCs. To investigate if GBM6 expresses stem-cell like characteristics, the gene expressions of GBM stem cells (GSCs) in GBM6 and two non-GSC glioma lines (U87 and U118) were examined and compared using qRT-PCR. GBM6 cell lines expressed a number of genes associated with GSC, including CD133, CD44, Nestin, Sox 2 and MGMT. Expression of these markers was appreciably elevated compared to those observed in U87 and U118. Further conducted experiments were conducted to verify if brain tumor tissues from GBM6 xenograft express characteristic markers of GSCs. GBM6 cells were passaged as xenografts and then inoculated in mouse brains. After 4 weeks when tumors grew to be detectable, the GBM6 tumors were harvested, sectioned, and fluorescently immunostained. The tumor sections were found to express GSC characteristic surface markers including CD44, Nestin2, and Sox-2 (FIG. 4A). That the GBM6 brain tissues exhibited readily detectable MGMT activity was confirmed.

To investigate if NP-siRNA-CTX preferentially targets tumors, mice bearing GBM6 tumors were administered with NP-siRNA-CTX via tail vein injection and mice receiving no injection served as control. Fluorescence images of mouse brains were acquired by Xenogen 3 hr post injection and NP-siRNA-CTX was found to accumulate in tumor but not in healthy brain tissue (FIG. 4B). The GBM6 tumors were then harvested, sectioned, imagined by fluorescence microscopy. NP-siRNA-CTX was found to accumulate adjacent to nuclei (DAPI stained), indicating the uptake of siRNA by tumor cells (FIG. 4C). Another set of tumor tissue sections were stained with Prussian blue to examine the localization of NP-siRNA-CTX. NP-siRNA-CTX (by iron oxide core) was seen in tumor parenchyma.

To examine the biological activity of NP-siMGMT-CTX, mice bearing GBM6 xenografts were randomly divided into three groups. Two groups of mice were intravenously injected with NP-siMGMT-CTX and NP-siGFP-CTX (non-therapeutic control), respectively. The other group was left untreated. In the treatment groups, each mouse received one injection per day for 4 days. The mice were the sacrificed and the xenografts collected 24 hr after the last injection. The MGMT expression (FIG. 4D) of brain tumor tissues was assessed by the western blot assay and the MGMT activity (FIG. 4E) of cleared whole tumor homogenates was evaluated by a standard biochemical assay as detailed previously (M. S. Bobola, M. S. Berger, R. G. Ellenbogen, T. S. Roberts, J. R. Geyer, J. R. Silber, Clinical Cancer Research 2001, 7, 613). As shown, the MGMT expression of xenografts from mice treated with NP-siMGMT-CTX was approximately 2-fold lower than those from untreated (β≤0.05) and siGFP-treated (β≤0.05) mice. Similarly, the xenograft MGMT activity of mice treated with NP-siMGMT-CTX was 1.7 to 2.1-fold lower than those of untreated (156±65 vs. 260±65 fmol/10⁶ cells; P≤0.0001) and siGFP-treated (156±65 vs. 331±87 fmol/10⁶ cells; P≤0.0001) mice. These findings demonstrate that NP-siMGMT-CTX effectively delivered functional siRNA to GBM6 tumor cells and suppressed their MGMT expression and activity in vivo.

Therapeutic Efficacy of NP-siMGMT-CTX in Orthotopic GBM Serially-Passaged Patient-Derived Xenografts

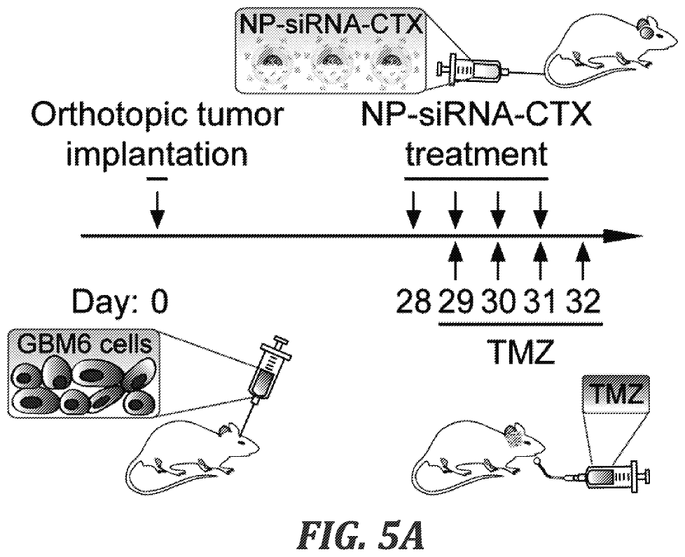

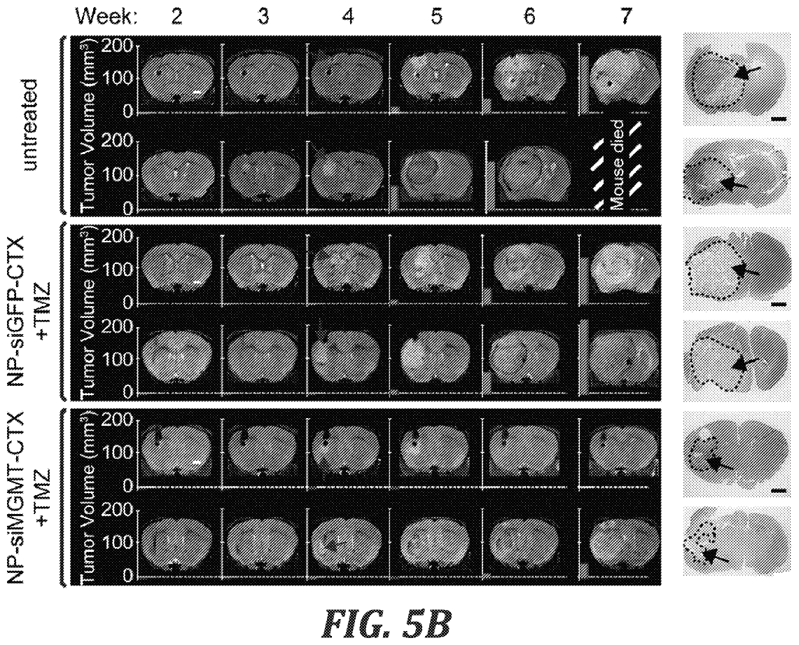

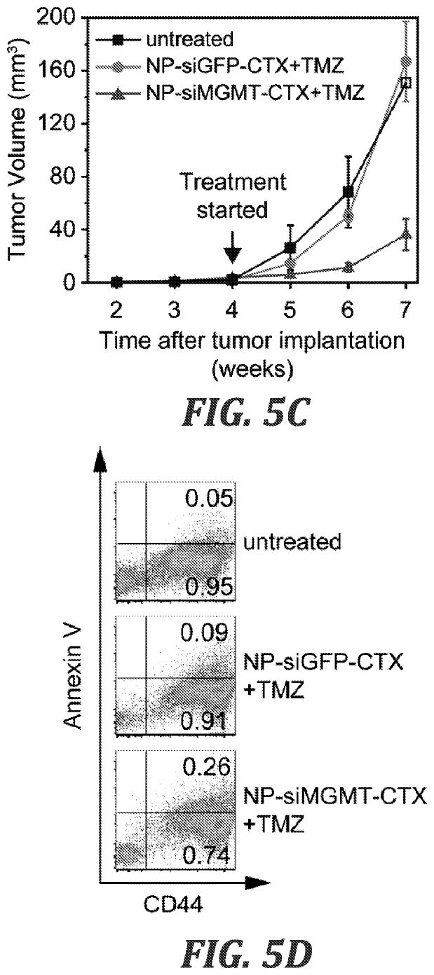

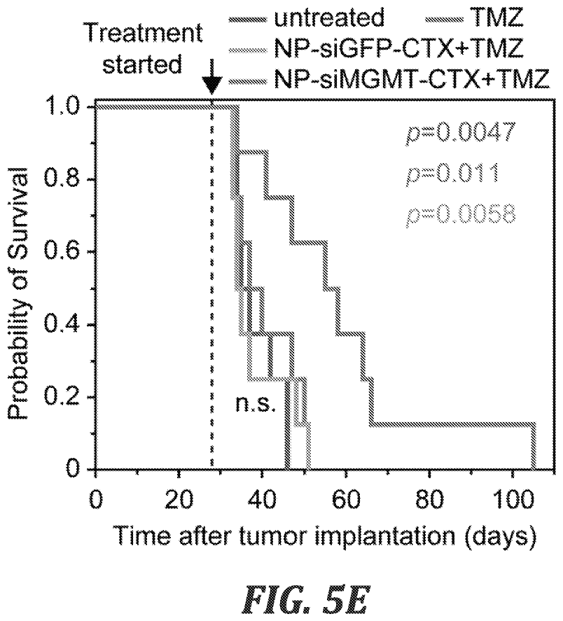

To determine whether greater sensitivity of tumor cells to TMZ would result from suppression of MGMT in GBM6 cells, xenograft growth, monitored by high-resolution MRI (14T), was compared between untreated mice and mice treated concurrently with TMZ and either NP-siMGMT-CTX or NP-siGFP-CTX. The treatment schedule is shown in FIG. 5A. Mice were inoculated with GBM6 cells intracranially (day 0). Four weeks after tumor inoculation (day 28), each mouse bearing GBM6 xenograft received 4 intravenous daily injections of either NP-siGFP-CTX or NP-siMGMT-CTX. The first dose of TMZ was given by oral lavage at the second injection, followed by three additional doses of TMZ given in the following three days, respectively.

The brains of mice treated with either NP-siMGMT-CTX or NP-siGFP-CTX and with TMZ were imaged by MRI and the tumor volumes were measured over a period of 7 weeks.

FIG. 5B shows the MRI images of mouse brains acquired at 2, 3, 4, 5, 6, and 7 weeks after tumor inoculation. Two mouse brains are displayed for each treatment condition (untreated: rows 1 and 2; treated with NP-siMGMT-CTX+TMZ: rows 3 and 4; treated with NP-siGFP-CTX+TMZ: rows 5 and 6). The last column on the right of FIG. 5B display optical images of H&E stained whole brain sections obtained at sacrifice. The tumor volume for each treatment is displayed by the red bar graph alongside corresponding images. The mice treated with NP-siMGMT-CTX and TMZ showed significant tumor volume reduction while mice treated with control NP-siGFP-CTX+TMZ and the untreated mice exhibited a similar trend of increase in tumor volume. At onset of TMZ treatment at week 4, tumors were detectable in all mice (red arrows), with an average size of 2.7 mm³. FIG. 5C shows the tumor volume of mice as a function of time, as evaluated from the MR images, for a period 7 weeks starting from tumor inoculation for mice treated under three treatment conditions. As shown, the tumor growth rate was similar between untreated and NP-siGFP-CTX+TMZ treated mouse groups and was significantly faster than the tumor growth rate of the mouse group treated with NP-siMGMT-CTX+TMZ. At week 6, the average tumor volume of the mice treated with NP-siMGMT-CTX+TMZ was 11±3.3 cm³, >5 times smaller than the average tumor volume (61±16 cm³) of the mice in the other two groups. Tumor volumes obtained by measurements of H&E stained xenograft sections of the three mice groups (the last column on the right of FIG. 5B) yielded the consistent results with MRI measurements. These results indicate that the treatment with siMGMT and TMZ significantly slowed down the tumor growth.

Whether the treatment with NP-siMGMT-CTX reduced the resistance of GSCs to TMZ was then determined. Mice bearing GBM6 tumors were intravenously injected with either NP-siMGMT-CTX or NP-siGFP-CTX, or received no injection as control. The mice were treated following the treatment schedule shown in FIG. 5A. Tumors were collected 3 days after the last TMZ treatment. The collected tumors were disintegrated to single cells that were double stained for Annexin V (labeled with Alexa 647), a marker of apoptosis, and CD44 (labeled with PE), a presumptive marker of GSCs. Fluorescently-labeled cells were then analyzed with flow cytometry. FIG. 5D shows that the majority of GBM6 tumor cells are CD44 positive, and tumor cells from mice treated with NP-siMGMT-CTX contained significantly more Annexin V⁺ and CD44⁺ cells than the tumor cells obtained from untreated and NP-siGFP-CTX treated mice. These findings provide evidence that NP-siMGMT-CTX was taken up by GSCs, a subpopulation of GBM cells implicated in promoting treatment resistance leading to tumor recurrence, and sensitized GSCs to TMZ leading to increased apoptosis.

To demonstrate the efficacy of NP-siMGMT-CTX on prolonging the survival of mice bearing GBM tumors, the mice were randomly divided into three groups with each group treated with one of three treatment conditions: NP-siGFP-CTX+TMZ, NP-siMGMT-CTX+TMZ, and TMZ only, following the treatment schedule shown in FIG. 5A. The mice were closely monitored during the entire treatment period until the time of euthanasia. FIG. 5E shows the survival of mice bearing GBM6 tumors as a function of time, determined by the method of Kaplan-Meier. As shown, the mice treated with NP-siMGMT-CTX showed significantly longer median survival (58.8±7.7 days, P<0.05) than untreated (38.5±1.9 days), TMZ treated (40.8±2.7 days), and NP-siGFP-CTX treated (38.1±2.5 days) mice. Mice treated with NP-siMGMT-CTX extended their lifespan for 18.3 days while mice treated with TMZ alone extended their lifespan for 2.3 days. This result indicates that NP-siMGMT-CTX in combination with TMZ prolonged survival by 8.8-fold compared to the treatment with TMZ only in a mouse model of glioblastoma serially-passaged patient-derived xenograft. Notably, one NP-siMGMT-CTX treated mouse had a tumor with a volume of 124 mm³ at week 3 and the tumor was undetectable at week 6 survived 80 days (see FIG. 5E). The result indicates that the GBM6 is highly resistant to TMZ and the treatment with combined siMGMT and TMZ greatly enhances the killing of GBM and GSC cells as compared to mice treated with TMZ alone. Together these results revealed a correlation between improved survival and reduction in tumor burden.

The Efficacy of Suppression of MGMT Expression and the Nature of the Association of Anti-MGMT siRNA to the Nanoparticle: Non-Covalently Associated siRNA Vs Covalently Coupled siRNA

The nanoparticle of the invention is advantageously effective in suppressing expression of O⁶-methylguanine-DNA methyltransferase (MGMT) by delivering biologically active siRNA to manipulate gene expression in human GBM and improve the clinical outcome for GBM. The success of the nanoparticle is due in significant part to the targeted and effective delivery of an siRNA effective to reduce the expression of MGMT.

As described herein, the nanoparticle of the invention consists of (a) an iron oxide core and a chitosan-polyethylene glycol-polyethylenimine copolymer coating surrounding the core; (b) chlorotoxin covalently coupled to the coating; and (c) an siRNA (e.g., anti-MGMT siRNA) reversibly associated to the coating by non-covalent interaction. The nanoparticle includes an siRNA reversibly associated with the nanoparticle's coating, which as defined herein, means that the siRNA is not covalently coupled to the nanoparticle. The non-covalent interaction is an associative interaction between the siRNA and the nanoparticle and is an ionic interaction, not covalent bonding. In the nanoparticle, the siRNA is associated to the nanoparticle's coating through a non-covalent interaction with the polyethylenimine (PEI) of the coating. The siRNA is stably associated with the nanoparticle as the nanoparticle trafficks the circulatory system and then is advantageously released from the nanoparticle once the nanoparticle reaches its cellular target (endosome) where the pH of the cell's endosome is effective to facilitate release of the siRNA from the nanoparticle.

The nanoparticle of the invention differs from other similarly constituted known nanoparticles. As described herein, in the nanoparticle of the invention the siRNA is associated to the nanoparticle by non-covalent interaction, which is in contrast to known nanoparticles where the siRNA is covalently coupled to the nanoparticle and is not readily released from the nanoparticle in environment of the endosome.

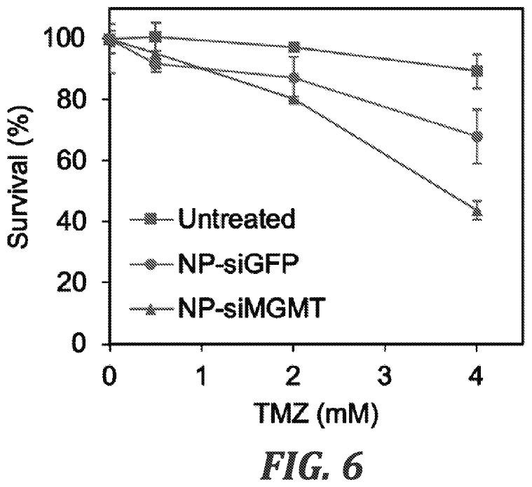

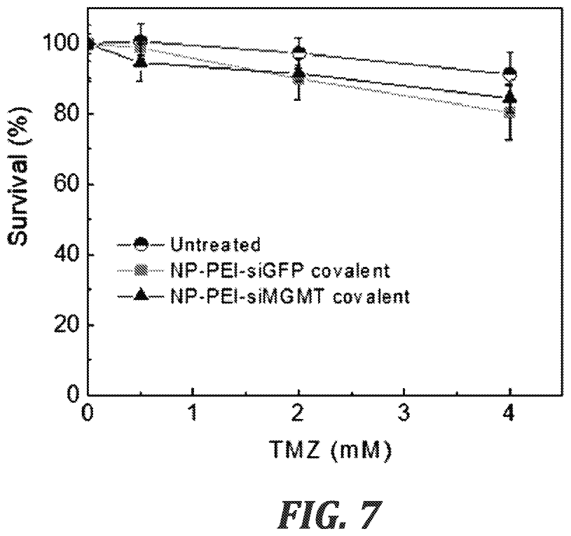

The survival curves for (viability vs. drug dose) of GBM6 cells treated with a nanoparticle of the invention with siRNA associated to the nanoparticle by non-covalent interaction and a similarly constituted nanoparticle with siRNA covalently coupled to the nanoparticle are presented in FIGS. 6 and 7, respectively.

FIG. 6 compares survival curves of GBM6 cells treated with NP-siGFP (“NP-siGFP complex”) in which siGFP is associated by non-covalently interaction to NP-PEI, NP-siMGMT (“NP-siMGMT complex”) in which siMGMT is associated by non-covalently interaction to NP-PEI, or left untreated.

Referring to FIG. 6, GBM6 cells were treated with NP-siGFP complex or NP-siMGMT complex for 48 hours or left untreated and then treated with 4 mM TMZ for additional 48 hours. GBM6 cells receiving no NP treatment displayed strong TMZ resistance after the TMZ treatment as demonstrated by near 90% remaining viability. In contrast, only 44% of GBM6 cells receiving NP-siMGMT treatment remained viable after TMZ treatment, which is significantly lower than the viabilities of the cells receiving no NP treatment and cells receiving NP-siGFP treatment. These results indicate that the treatment with NP-siMGMT in combination with TMZ significantly improves GBM6 cell killing.

FIG. 7 compares survival curves of GBM6 cells treated with NP-PEI-siGFP covalent (siRNA covalently coupled to the NP), NP-PEI-siMGMT covalent (siRNA covalently coupled to the NP), or left untreated. siGFP and siMGMT are covalently bound to the nanoparticles with a PEI coating (see Biomaterials, 32 (24), 5717-5725 (2011)).

GBM6 cells were treated with either nanoparticles loaded with siMGMT for 48 hours or left untreated and were subsequently treated with 4 mM TMZ 48 hrs. 84% of GBM6 cells treated with NP-PEI-siMGMT remained viable after TMZ treatment, which is similar to the viability of cells treated with NP-PEI-siGFP (80%) and lower than the viability of cells receiving no NP treatment (91%). This result indicates that the treatment with NP-PEI-siMGMT in combination with TMZ does not improve the therapeutic efficacy in cell killing as compared with NP-PEI-siGFP and untreated group. The slightly lower viability of GBM6 cells treated with NP-PEI-siMGMT and NP-PEI-siGFP than the viability of untreated cells is likely due to the minor toxicity caused by the nanoparticles.

As FIGS. 6 and 7 make clear, TMZ in combination with the nanoparticles of the invention having siRNA associated to the nanoparticle by non-covalent interaction are therapeutically efficacious in GBM cell killing, while the combination of TMZ with the nanoparticles having siRNA covalently coupled to the nanoparticle are not.

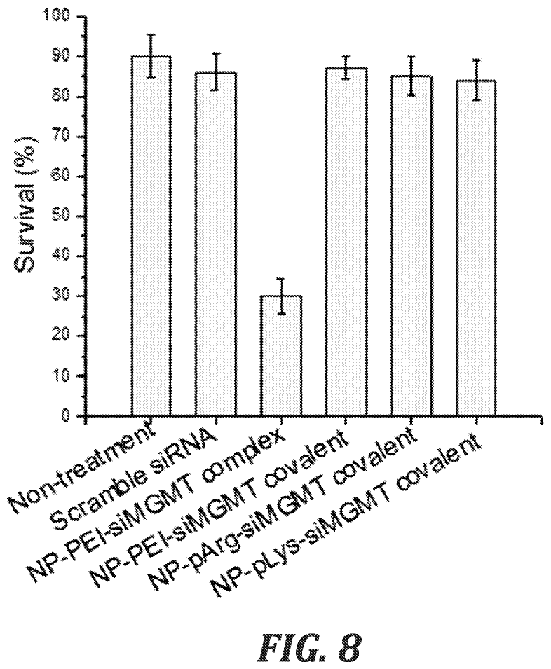

The viability of GBM6 cells treated with a nanoparticle of the invention with siRNA associated to the nanoparticle by non-covalent interaction and a similarly constituted nanoparticle with siRNA covalently coupled to the nanoparticle are presented in FIG. 8.

FIG. 8 compares the viability of GBM6 cells treated with various nanoparticle formulations including (as shown on horizonal axis) NP-PEI-siMGMT complex, NP-PEI-siMGMT covalent, NP-pArg-siMGMT covalent, NP-pLys-siMGMT covalent and control cells (Non-treatment: cells receiving no NP treatment, Scramble siRNAs: cells treated with scramble siRNA). In NP-PEI-siMGMT complex, siMGMT was complexed onto NP-PEI. Scramble siRNAs refers to the siRNAs with the molecular weight similar to siMGMT but with different gene sequence and are used as a negative control. In NP-PEI-siMGMT covalent, NP-pArg-siMGMT covalent, and NP-pLys-siMGMT covalent, siMGMTs are covalently coupled to the NP coated with polyethyleneimine (PEI), polyarginine (pArg), polylysine (pLys) coatings, respectively, (see Biomaterials, 32 (24), 5717-5725 (2011).

The preparation of the nanoparticles having covalently coupled siRNA is described in Zhang et al., “Chlorotoxin bound magnetic nanovector tailored for cancer cell targeting, imaging, and siRNA delivery”, Biomaterials, 31 (31), 8032-8042 (2010).

GBM6 cells were treated nanoparticles of various formulations (see caption of FIG. 8) for 48 hours or left untreated and then treated with 4 mM TMZ for additional 48 hrs. Among all the cell groups, only the cell group treated with NP-PEI-siMGMT complex showed significantly reduced viability (about 30%) after the TMZ treatment. Cells treated nanoparticles with covalently bonded siMGMT (i.e., NP-PEI-siMGMT covalent, NP-pArg-siMGMT covalent, and NP-pLys-siMGMT covalent) showed about 85% remaining viability, similar to or slightly lower than the viabilities of cells receiving no NP treatment (90%) and cells receiving Scramble siRNA treatment (85%). These results indicate that covalently attached siMGMT on nanoparticles coated with a polymer coating (PEI, pArg, or pLys) is unable to sensitize cancer stem cells to TMZ for effective cell killing. Similar results (i.e., negligible cell killings) of these covalent nanoparticle formulations were also demonstrated in the studies with other cancer stem cells including GBM8, GSC38b, GSC41b, and GBM6 tumors (not shown). It has been shown that it is much more challenging to sensitize cancer stem cells to TMZ than regular GBM cell lines with siMGMT. This is likely due to the fact that cancer stem cells are much more resistant to TMZ than GBM cell lines and require a much higher dose of TMZ to treat effectively than cells from GBM cancer lines. For instance, GBM6 (cancer stem cell) requires 4 mM TMZ, and SF763 and A1235 (GBM cell lines) require only 400 μM and 10 μM TMZ, respectively for effective treatment (see FIGS. 2A-2J). The amount of siRNAs covalently bonded to NPs can be limited due to the limited covalent bonding sites available on the nanoparticle surface and may not be enough to sensitize cancer stem cells to TMZ for effective cell killing. In contrast, NP-PEI-siMGMT complex can be loaded with and deliver a much greater amount of siRNAs than nanoparticle covalent formulations, and thus shows much higher effectiveness in sensitizing cancer stem cells and better therapeutic effect in cell killing.

As FIG. 8 makes clear, TMZ in combination with the nanoparticles of the invention having siRNA associated to the nanoparticle by non-covalent interaction are therapeutically efficacious in GBM cell killing, while the combination of TMZ with the nanoparticles having siRNA covalently coupled to the nanoparticle are not.

Advantages of the NP-siRNA-CTX System

MGMT is a potent resistance factor to contemporary concurrent TMZ-radiotherapy as evidenced by the strong inverse correlation between prolonged survival rates and very low or absent MGMT expression accompanying MGMT promoter methylation in a minority of GBMs. _ENREF_27 These observations emphasize the need for clinically tractable strategies to suppress MGMT-mediated resistance in order to improve clinical outcome. The nanoparticle formulation described herein is suitable for systemic delivery of anti-MGMT siRNAs by incorporating PEI to the CP polymer to bind siRNA. The resulting particle, NP-siMGMT-CTX, bound about 300 siRNA molecules per nanoparticle and retained the favorable characteristics of CP-CTX that facilitate the uptake by tumor cells and intracellular release of functional therapeutic ligand. Other advantages of NP-siMGMT-CTX include binding capacity that approximates the number of siRNA needed to for RISC to produce a therapeutic effect and absence of discernible toxicity (see FIGS. 3A-3N).

Importantly, the data indicate that CP-PEI polymer protects siRNA against degradation by serum nucleases, a major impediment to the clinical translation of siRNA therapeutics. Serum contains a variety of RNases that are markedly elevated in many malignancies, including at least one activity that degrades double-stranded RNAs. Results suggest that CP polymer dissolution occurs in the endosome, possibly exposing siRNA to degradative enzymes. Binding of siRNA to PEI is unaffected by polymer breakdown and protects against degradation during transport to the RISC.

NP-siMGMT-CTX significantly increased TMZ sensitivity in both cultured GBM cells in vitro (see FIGS. 2A-2J) and GBM6 xenografts in vivo (see FIGS. 4A-4E). Sensitization of GBM6 to TMZ was comparable to that produced by intra-tumoral injection of free or NP-CP bound BG. _ENREF_35 This result is notable as NP-siMGMT-CTX reduced MGMT activity by only 40 to 50% (see FIGS. 4A-4E) prior to exposure to TMZ. It is likely that multiple, serial treatments with NP-siMGMT-CTX were responsible, at least in part, for significantly lengthening survival. Longer survival may also reflect sensitization of GBM6 cells expressing stem-like markers, a subpopulation believed responsible for treatment resistant. While the survival of mice treated with NP-siMGMT-CTX without TMZ was not tested, MGMT knockdown itself would not be expected to increase the survival. Rather, patients with low MGMT expression are associated with improved survival following alkylator therapy_ENREF_13. Furthermore, possible toxicity from the NP itself was tested with the control (NP-siGFP-CTX)+TMZ treatment condition, which showed no difference in survival as compared to TMZ alone indicating the biocompatibility of the NPs.

The ability to give NP-siMGMT-CTX intravenously, common practice for anti-tumor drug delivery, facilitates clinical application. Importantly, the iron oxide core and CP polymer of NP-siMGMT-CTX are biocompatible, biodegradable, will be rapidly cleared from the circulation, and have features that should limit toxicity. Low toxicity suggests NP-siMGMT-CTX can be given with TMZ, especially during daily radiotherapy. Ablating MGMT activity synergistically increases radiosensitivity in human GBM cell lines treated concurrently with minimally cytotoxic TMZ doses. More recently, it has been shown that less than the median level of MGMT activity in promoter unmethylated GBMs is associated with significantly longer progression-free survival following alkylator therapy. These findings indicate that intervention to suppress MGMT expression would increase the efficacy of both the concurrent and adjuvant component of standard TMZ therapy in promoter unmethylated GBMs. Note that therapeutic efficacy of the nanoparticle can be further increased by the decrease in particle size, increase in the dose of siRNAs, and optimization of scheduling in siRNA and TMZ.

The ability of NP-siMGMT-CTX to deliver biologically active siRNA provides a novel method to manipulate gene expression in human GBM. There are a large number of DNA repair activities that are attractive targets to circumvent radiation and drug resistance. Nanoparticles could also deliver siRNA targeting essential tumor pathways regulating metabolism, angiogenesis, oxidative stress, migration and invasion, and suppression of immune response. Employing therapeutic siRNAs, in addition to minimizing non-specific effects, simplifies nanoparticle design, and eliminates modification of chemical agents to permit binding and controlled release. Utilizing CP polymer conjugated with CTX facilitates BBB penetration, an essential feature for any therapeutic agent targeting GBM cells that have diffusely infiltrated surrounding brain as well as bulk tumor. CTX displays high avidity and specificity for GBM compared to normal brain tissue and targets the majority of primary brain tumors examined (74 out of 79). It has been shown that NPs coated with CP polymer conjugated with CTX are able to cross the BBB and retained in tumor tissue, but quickly clear from normal brain where there is a lack of specific cell binding by CTX. It has also been shown that some NPs are able to induce endothelial cell leakiness (NanoEL) because of their size and density. Here, the specific targeting at the cellular level offered by CTX combined with the specificity in silencing single expression at the molecular level provided by siRNA would greatly reduce off-target toxicity and morbidity, a formidable challenge of clinical chemo- and radiotherapy. Such attributes suggest that siRNA delivered by the NPs of the invention can improve clinical outcome for GBM.

The present invention provides _ENREF_38 a simple, effective, and multifunctional nanoparticle formulation with many unique features for effective siRNA delivery and GBM treatment. The nanoparticles can also serve as an MRI contrast agent for tumor imaging and monitoring of response to drug. Monodispersed iron oxide cores with ultra-fine size and special surface coatings ensure the small size of the final nanocarriers (<60 nm), which facilitate BBB penetration, high tumor accumulation, uptake by endocytosis, and trafficking to intracellular targets. The nanoparticles of the invention show, no apparent myelo-, hepato-, renal, or gross tissue toxicities in mice, and display only short-term retention in healthy tissues. The nanoparticles are able to facilitate siRNA loading, protect siRNA from degradation, confer tumor specificity, and overcome extra- and intra-cellular barriers (including the BBB). More importantly, the nanoparticles markedly enhance the killing of drug-resistant GBMs and GSCs as compared to TMZ alone, and significantly extend the survival of mice bearing serially-passaged patient derived GBM6 orthotopic xenografts.

As used herein, the term “about” refers to ±5% of the specified value.

### Experimental Methods

CP-PEI copolymer synthesis. PEG was grafted to depolymerized chitosan using a method described previously (N. Bhattarai, H. R. Ramay, J. Gunn, F. A. Matsen, M. Zhang, J Controlled Release 2005, 103, 609). Amine groups on 50 mg/ml 25,000 Da polyethylenimine (PEI, Sigma-Aldrich) were modified with 2-iminothiolane (Traut's reagent, Molecular Biosciences) for 1 hr at room temperature in thiolation buffer (pH 8.0) at 1:1.2 molar ratio. Concurrently, 20 mg/ml chitosan-grafted PEG (CP) was modified with N-succinimidyl iodoacetate (SIA, Molecular Biosciences) at a 1:10 molar ratio at room temperature in thiolation buffer (pH 8.0) through N-hydroxysuccinimide ester chemistry before removing unreacted SIA reagent using a Zeba spin column (Thermo Fisher Scientific) equilibrated with thiolation buffer (pH 8.0). The modified CP was then added to PEI-Traut's for attachment through the formation a thioether bond. After reaction at room temperature for 4 hr, the resultant mixture was dialyzed with a dialysis membrane (MW 50,000 cut, Spectrum Labs) against distilled water for 2-3 days, and the solution was subsequently freeze-dried.

CP-PEI copolymer characterization. The presence of the constituent polymers on the lyophilized CP-PEI was verified by proton NMR (¹H-NMR) in D₂O. Trimethylsilyl propionate was used as the internal standard. NMR spectra of polymer coatings were obtained using a Bruker Avance 301 spectrometer operating at 300 MHz and 298 K.

Nanoparticle synthesis. Synthesis of iron oxide nanoparticles with a siloxane poly(ethylene glycol) (PEG) monolayer (IOSPM) was conducted with a method described previously (C. Fang, N. Bhattarai, C. Sun, M. Zhang, Small 2009, 5, 1637). The IOSPM has unique properties including superparamagnetism, small and uniform size, high stability in biological solutions, and excellent biodegradability. CP-PEI was conjugated to IOSPM through thiol-ester chemistry (see FIG. 1A). In brief, 50 mg/ml CP-PEI was first functionalized with iodoacetyl groups using SIA at a 1:10 molar ratio at room temperature. The excess of SIA was purified using a Zeba spin column equilibrated with thiolation buffer (pH 8.0). 1 mg/ml IOSPM was simultaneously reacted with Traut's reagent at room temperature for 1 hr; then combined with the purified CP-PEI-SIA overnight before purified through Zeba column equilibrated with 20 mM HEPES buffer (pH 7.4).

NP-siRNA-CTX preparation. NP and siRNA were mixed in 20 mM HEPES buffer (pH 7.4) for 30 min to allow formation of NP-siRNA complexes. Afterwards, a 1 mg/mL solution of CTX was thiolated through reaction with Traut's reagent at a 1.2:1 molar ratio for 1 hr in the dark at room temperature. Concurrently, SIA was conjugated to amine functional groups on NP at 1 mg of SIA/mg iron in the dark with gentle rocking for 1 hr. Subsequently the thiolated CTX was reacted with the iodoacetyl groups on the SIA at 1 mg CTX per 0.9 mg Fe, anchoring CTX to the surface of the siRNA-bound NP to form NP-siRNA-CTX.

To evaluate the degree of CTX attachment to NPs, NP-siRNA-CTX were prepared as described above after purification of unbound CTX through S-200 Sephacryl resin (GE Healthcare Life Sciences). Both purified and unpurified NP-siRNA-CTX were boiled in loading buffer containing 10% 2-mercaptoethanol. Released or unreacted CTX from both reduced and un-reduced NP-siRNA-CTX were separated from NP-siRNA-CTX through SDS-PAGE and quantified using the Quantity One software package (Bio-Rad) and a standard curve of CTX at known concentrations.

NP-siRNA complex characterization. siRNA binding and serum stability were characterized using native polyacrylamide gel electrophoresis (PAGE, Bio-Rad). NP-siRNA complexes were formed as described previously at concentrations corresponding to the W/W ratios (iron weight of NP/siRNA weight) tested (0.5:1, 1:1, 2:1, 5:1, 10:1 and 20:1). While maintaining a uniform concentration of siRNA, NP-siRNA complexes were prepared at NP:siRNA weight ratios ranging from 0:5 to 20:1. The complex were treated with heparin (1000 units/ml, 50 μL heparin/1 μg siRNA) and incubated for 30 min at room temperature to block the electrostatic interaction between NP and siRNA. Both heparin treated and untreated samples were loaded onto the gel and electrophoresis for about 30 min at 120 V. Gels were stained with 0.5 μg/ml ethidium bromide and visualized using a Bio-Rad Universal Hood II Gel Doc System.

For serum stability assay, the NP-siRNA complex at a series of weight ratios of NP to siRNA as mentioned above were incubated in a 10:1 volume ratio of medium and fresh serum to give 10% serum concentration and incubated at 37° C. overnight. Then the complexes were treated with heparin before running the polyacrylamide gel. Naked siRNA served as the control. All characterization studies were repeated with at least three independently prepared batches of NPs.

Cell culture. All tissue culture reagents were purchased from Life Technologies unless specified otherwise. The MGMT-proficient SF763 (activity=59±3 fmol/10⁶ cells, i.e., 35,400±1,800 molecules/cell) (M. S. Bobola, S. Varadarajan, N. W. Smith, R. D. Goff, D. D. Kolstoe, A. Blank, B. Gold, J. R. Silber, Clin Cancer Res 2007, 13, 612) and MGMT-deficient A1235 human GBM (M. S. Bobola, D. D. Kolstoe, A. Blank, J. R. Silber, Mol Cancer Ther 2010, 9, 1208; and R. S. Day, 3rd, C. H. Ziolkowski, D. A. Scudiero, S. A. Meyer, A. S. Lubiniecki, A. J. Girardi, S. M. Galloway, G. D. Bynum, Nature 1980, 288, 724) cell lines were cultured at 37° C. in a humidified atmosphere with 5% CO₂ using Dulbecco's modified Eagle's Medium (DMEM) supplemented with 10% fetal bovine serum (Atlanta Biologicals) and 1% antibiotic-antimycotic. SF767 was obtained from the Brain Tumor Research Center at University of California, San Francisco (UCSF). A1235 cells were obtained from the University of Alberta. U-87 MG (U87) and U-118 MG (U118) cells were purchased from ATCC and maintained in DMEM supplemented with 10% fetal bovine serum (Atlanta Biologicals) and 1% antibiotic-antimycotic.

In vitro cell transfection. Twenty-four hours after plating cells at 30,000 cells per well in 24 well plates, cells were transfected with NP-siRNA at 100 nM of siRNA. After incubation for 24 hr, the transfection medium was replaced with fresh medium, and cells were incubated for an additional 48 hr before analysis. The siRNA targeting green fluorescence protein (GFP) and the ON-TARGET plus siRNA against MGMT (siMGMT) were purchased from GE Dharmacon.

NP uptake and intracellular trafficking. To evaluate uptake and intracellular distribution of NP-siRNA, NP was conjugated with Cyanine3 (Lumiprobe), and complexed with Dy677 labeled siScrambled. Cells were transfected with NP-siRNA as described above. At 6, 24, 48, and 96 hr of incubation, the LysoTracker Green probe was applied to label endosome following the manufacturer's instructions (Thermo Fisher Scientific). Hoechst 33342 (1 μg/ml, Biotium) was used to stain nuclei. Cells were observed and imaged using a Nikon ECLIPSE TE2000-S microscope.

Quantitative RT-PCR (qRT-PCR). RNA was extracted from cells at 72 hr after siRNA transfection using the Qiagen RNeasy kit. cDNA was prepared using the iScript cDNA Synthesis Kit (Bio-Rad) following the manufacturer's protocol, which was then used as a template for PCR. qRT-PCR was used to evaluate the relative expression levels of MGMT utilizing human β-actin as a reference gene. SYBR Green PCR Master mix (Bio-Rad) was used for template amplification with a primer for each of the transcripts in a Bio-Rad CFX96 real-time PCR detection system. Quantitative amplification was monitored by the level of fluorescence reflecting the cycle number at the detection threshold (crossing point) using a standard curve. Thermocycling for all targets was carried out in a solution of 20 μl containing 0.5 μM primers and 4 μg of cDNA from the reverse transcription reaction under following conditions: 95° C. for 2 min, 40 cycles of denaturation (15 sec, 95° C.), annealing (30 sec, 58° C.), and extension (30 sec, 72° C.). The primers used for β-actin is: 5′-AGCGAGCATCCCCCAAAGTT-3′ (SEQ ID NO: 1)/5′-GGGCACGAAGGCTCATCATT-3′ (SEQ ID NO: 2). The pre-evaluated primers used for MGMT amplification was purchased from Bio-Rad.

Cell survival assays. Alamar blue assay was used to evaluate cell viability following the manufacture's protocol (Life Technologies). Briefly, GBM cells were plated and transfected with NP-siRNA at 100 nM of siRNA as described above. AB assay was conducted at 2 days after transfection. Cells were washed with PBS (pH 7.4) three times before adding 10% AB in complete growth medium to the wells. After incubation for 1 hr, the AB solution was transferred to a 96-well plate, and fluorescent emissions at an excitation wavelength of 550 nm and an emission wavelength of 590 nm were read on a SpectraMax M5 microplate reader (Molecular Devices).

Clonogenic assay was used to evaluate cell response to drugs. Briefly, GBM cells were treated with control or siMGMT at 100 nM of siRNA for 48 hr as described above. The cells were then trypsinized, and 250-300 cells were plated per well in six-well plates in normal 10% FBS-containing growth medium in triplicate. Cells were then treated with drugs at 24 hr after plating and cultured for 1-2 weeks before fixation and staining with Methylene Blue (Sigma-Aldrich). Colonies consisting of 50 or more cells were counted. LD₅₀ was calculated from the dose response kill curve derived from the replicate assays.

In vivo biodistribution and serum half-life of NP. All animal experiments were conducted in accordance with University of Washington Institutional Animal Care and Use Committee (IACUC) approved protocols (IACUC 3441-05) as well as with federal guidelines. C57BL/6J WT mice (Jackson Laboratories) were systemically administrated via tail vein injection with Dy677 labeled NP-siScrambled-CTX (NP-siScr-Dy677-CTX as a siRNA control NP). The non-injected animal was included in the study as control. Two and or forty-eight hours after injection, the mice were euthanized and tissues were dissected from brain, liver, kidneys and spleen. Each tissue was imaged using a Xenogen IVIS Lumina II system (Perkin Elmer) to examine NP distribution.

Blood was collected by retro-orbital eye bleed or terminal heart puncture for blood half-life evaluation at 0.5, 1, 2, 4, 24, and 48 hr after NP injection. Due to the limited amount of blood from each animal, none were used for more than two time points. Blood samples were drawn from four independent mice for each time point. Blood (100 μl) was added to a 96 well clear bottom plate and scanned on the Odyssey NIR fluorescence imaging instrument (LI-COR) using the 700 nm-channel (λ_{exc}=685 nm with λₑₘ=705 nm). The concentration of NP was interpolated from the NP-siScr-Dy677-CTX fluorescence standard curve.

Immunofluorescence. Mice brains were collected and fixed in 4% formaldehyde for 24 hr before placed in 30% sucrose until fully saturated. Afterwards, the brains were frozen in OCT embedding medium (Sakura) at −80° C. before sectioned using a cryostat (Leica Biosystems). Frozen sections (5-10 μm thickness) were stained with primary antibodies (anti-CD31, MGMT, CD44, Nestin, Sox-2) (Abcam) followed by fluorophore conjugated secondary antibody (Abcam), and DAPI was used for nuclei staining. Brain sections were then photographed under a Zeiss 510 META confocal microscope.

In vivo toxicity studies. Wild-type (C57BL/6J WT) mice were injected with NP-siMGMT-CTX through tail vein at 10 μg siRNA daily for 4 days. Twenty-four hours after administration of the last dose, the mice were sacrificed and blood was collected by cardiac puncture. Blood samples were delivered to Phoenix Central Laboratory for hematology analysis. Representative organ tissues from each group were excised and fixed in 10% (v/v) formalin saline and processed for routine histopathological procedures. Paraffin embedded specimen were cut into 8 μm sections and stained with hematoxylin and eosin (H&E) for histopathological evaluations.

Orthotopic xenograft tumor model and therapy response experiments. All xenograft therapy evaluations were conducted using a GBM6 orthotopic tumor model. The human GBM6 tumor was provided by the Mayo Clinic. The procedure for establishing intracranial tumors was described previously (B. L. Carlson, J. L. Pokorny, M. A. Schroeder, J. N. Sarkaria, Current Protocols in Pharmacology 2011, 52, 1; B. L. Carlson, P. T. Grogan, A. C. Mladek, M. A. Schroeder, G. J. Kitange, P. A. Decker, C. Giannini, W. Wu, K. A. Ballman, C. D. James, J. N. Sarkaria, International Journal of Radiation Oncology, Biology, Physics 2009, 75, 212). In brief, flank GBM6 tumor xenografts were harvested, mechanically disaggregated and stereotaxically injected into the frontal lobe of 6-week-old female NOD-SCID mice (Jackson Laboratories). The injection coordinates are as follows: 3 mm to the right of the midline, 2 mm anterior to the coronal suture and 3 mm in depth. Each animal received 2 μL single injection of approximately 1×10⁶ cells. At about 4 weeks post-tumor implantation, mice with similar tumor size as evaluated by MRI were randomized to one treatment group treated with NP-siMGMT-CTX+TMZ, and three control groups treated with either NP-siGFP-CTX+TMZ, TMZ only, or untreated. 100 μl NP-siRNA-CTX via tail vein at a concentration that allowed for 10 μg siRNA per mice was performed daily for 4 days (See FIG. 5A). Temozolomide (TMZ, TCI AMERICA) was administered by oral gavage (100 mg/kg mouse weight daily, with methyl cellulose used as the carrier, dose chosen based on previous work with similar animal models) starting from the second day of the NP-siRNA-CTX injections. All mice used for therapy response evaluations were observed daily and euthanized at the time of reaching a moribund condition.

In vivo knockdown of MGMT. MGMT suppression was evaluated by western blot and MGMT activity assays. In brief, mice bearing GBM6 intracranial tumors were injected with NP-siGFP-CTX/NP-siMGMT-CTX through tail vein at 10 μg siRNA daily for 4 days. Untreated mice served as control. Twenty-four hours after the last injection, mice were euthanized and tumors were isolated and flash froze in liquid N₂.

For western blot assay, tumor tissues were solubilized by incubation for 15 min on ice in 0.1% Triton X-100 in PBS (pH 7.4). Tumor lysis was diluted 1:1 with Laemmli sample loading buffer containing 2% β-mercaptoethanol. After heating at 100° C. for 5 min, 10 μg of extract protein was resolved by SDS-PAGE and transferred onto nitrocellulose membranes. Membranes washed three times with TBS were incubated with 3% QuickBlocker (Chemicon) in TBS for 1 hr at room temperature and then incubated overnight at 4° C. with 1 μg/mL antibody against MGMT or j-actin (Abeam) in TTBS containing 3% QuickBlocker. Membranes were washed with TTBS before being incubated for 1 hr at room temperature with alkaline phosphatase-conjugated goat anti-rabbit secondary antibody (Bio-Rad) diluted 1:3000 in 3% QuickBlocker. Membranes were then washed thrice with TTBS and antibody binding visualized by chemiluminescence (Immun-Star detection kit; Bio-Rad) and quantified using the ChemiDoc system running the Quantity One software package (Bio-Rad).

The MGMT activity assay was conducted using a standard biochemical assay that quantifies the transfer of radioactivity from a DNA substrate containing [methyl³H]O⁶-methylguanine (specific activity, 80 Ci/mmol) to protein as detailed previously (M. S. Bobola, M. Alnoor, J. Y. S. Chen, D. D. Kolstoe, D. L. Silbergeld, R. C. Rostomily, A. Blank, M. C. Chamberlain, J. R. Silber, BBA Clinical 2015, 3, 1). Frozen tissues were homogenized followed by sonication in isotonic buffer. DNA was quantified in crude homogenates by the diphenylamine method that measures deoxyribose following degradation of DNA with heat and acid. The homogenates were cleared by centrifugation at 10,000×g for 30 min. Activity was normalized to cell number using a conversion factor of 6 μg DNA per human cell. All activities are the mean of at least 4 determinations that generally differed by no more than 20%.

In vivo tumor targeting evaluation. For evaluation of tumor targeting, mice were administrated with NP-siScr-Dy677-CTX by tail vein injection. Three hours post injection, brains were collected and imaged using Xenogen IVIS system. Representative brain tissues were processed for frozen sections and stained with DAPI as described previously. Brain slides were imaged using a Nikon ECLIPSE TE2000-S microscope.

In vivo apoptosis analysis. The in vivo response to the combinatory treatment of NP-siMGMT-CTX and TMZ was assessed by determining the level of apoptosis in the tumors using Annexin V assay. Mice with intracranially established GBM6 tumor xenografts were treated following the indicated treatment schedules given in FIG. 5A, and tumors were isolated on 3 days after the last TMZ treatment. Tumor cells were dissociated and passed through a 70 m cell strainer (Thermo Fisher Scientific) to acquire single cell suspension. They were then subjected to double staining with GSC marker, anti-CD44-PE (Abcam) and Annexin V (Life Technologies) following the manufacturer's protocol and analyzed by flow cytometry using a BD FACS Canto flow cytometer. All data were analyzed with Flow Jo.

MRI. Imaging acquisition was started at 2 weeks after tumor implantation. Mice were anesthetized with isoflurane (Piramal Healthcare), and positioned in an MRI-integrated respiratory system, with nose-cone for anesthetic and oxygen delivery, ear bar head holder, circulating bath for temperature control, abdominal pad for respiratory monitoring (SA Instruments; MR-compatible small animal monitoring and gating system), and vacuum line for expiratory gas extraction. MR images to determine tumor volumes were obtained once per week, up to 7 weeks after tumor implantation. Total session time under anesthesia per mouse was about 15 min. Mice were divided into treatment groups based on maximum tumor width at 4 weeks, with partitioning performed to equilibrate average pre-treatment tumor size between groups. Images were acquired on a Bruker 14 Tesla vertical-bore imaging system (Ultrashield 600 WB Plus), using a 25 mm single-channel ¹H radiofrequency receiving coil (PB Micro 2.5). A T2-weighted rapid acquisition with refocused echoes (RARE) sequence was used with the following parameters: TR/TE=4000/27 ms, in-plane resolution=52×78 μm², matrix 384×256. 2-D slices of 0.5 mm thickness were used. 14 slices were obtained for each mouse and centered on the tumor volume as visualized on a localizer sequence. Scanning time for the T2-weighted sequence was about 4 min. Manual regions of interest (ROIs) surrounding the external tumor margin were generated on a slice-by-slice basis using Bruker ParaVision imaging software under high-magnification of the acquired images. ROIs were produced by users blinded to the treatment group of each mouse, and inter-user comparison between manually generated ROIs yielded a volume estimate difference <5%. Scans with reduced slice thickness of 0.2 mm were also obtained on individual tumors, to compare the variability size estimation of small and large tumors using the chosen 0.5 mm slice thickness.

Statistical Analysis. For statistical analysis, acquired data were expressed as mean±SD. Statistical significance was determined using the Student's t-test. Significant values were designated as follows: P≤0.05. Statistics for overall survival in mouse studies was calculated with the log-rank (Mantel-Cox) test. Statistical significance was established at P≤0.05. Sample sizes of n=8 used in animal experiments were based on our previous experience with similar experimental set-ups and previously results such that appropriate statistic tests could yield significant results.

While illustrative embodiments have been illustrated and described, it will be appreciated that various changes can be made therein without departing from the spirit and scope of the invention.

1. A nanoparticle for targeted siRNA delivery, comprising:
(a) an iron oxide core and a chitosan-polyethylene glycol-polyethylenimine copolymer coating surrounding the core;
(b) chlorotoxin covalently coupled to the coating; and
(c) an siRNA reversibly associated to the coating by non-covalent interaction.

2. A nanoparticle for targeted siRNA delivery, consisting of:
(a) an iron oxide core and a chitosan-polyethylene glycol-polyethylenimine copolymer coating surrounding the core;
(b) chlorotoxin covalently coupled to the coating; and
(c) an siRNA reversibly associated to the coating by non-covalent interaction.

3. The nanoparticle of claim 1, wherein the iron oxide core has a size from about 4 to about 12 nm.

4. The nanoparticle of claim 1, wherein the nanoparticle has a size from about 35 to about 80 nm.

5. The nanoparticle of claim 1, wherein the chlorotoxin is present in an amount from 1 to about 200 chlorotoxins per nanoparticle.

6. The nanoparticle of claim 1, wherein the siRNA is present in an amount from about 100 to about 400 siRNAs per nanoparticle.

7. The nanoparticle of any-one-of-claims-1-6 claim 1, wherein the siRNA reduces the expression of O⁶-methylguanine-DNA methyltransferase in a subject.

8. The nanoparticle of claim 1, wherein the siRNA is siMGMT.

9. A pharmaceutical composition comprising the nanoparticle of claim 1 and a pharmaceutically acceptable carrier.

10. The pharmaceutical composition of claim 9 formulated for intravenous injection.

11. A method for suppressing the expression of O⁶-methylguanine-DNA methyltransferase in a subject, comprising administering an effective amount of a nanoparticle of claim 7 to a subject in need thereof.

12. A method for treating brain cancer in a subject, comprising administering a therapeutically effective amount of a nanoparticle of claim 7 to a subject in need thereof.

13. The method of claim 12, wherein the brain cancer is a glioblastoma.

14. The method of claim 12, wherein the brain cancer is glioblastoma multiforme.

15. A method for killing cancer stem cells in a subject, comprising administering a therapeutically effective amount of a nanoparticle of claim 7 to a subject in need thereof.

16. A method for magnetic resonance imaging a tumor in a subject, comprising administering an effective amount of a nanoparticle of claim 1 to a subject in need thereof.

17. The method of claim 11, wherein the nanoparticle is administered intravenously.

18. The method of claim 11, wherein the subject is a human.

19. A method for making a nanoparticle for targeted siRNA delivery, comprising contacting an siRNA with an iron oxide nanoparticle, wherein the iron oxide nanoparticle comprises an iron oxide core and a chitosan-polyethylene glycol-polyethylenimine copolymer coating surrounding the core, and a targeting agent covalently coupled to the coating.

20. The method of claim 19, wherein the targeting agent is chlorotoxin.

21. (canceled)
